(19) United States
(12) Patent Application Publication
(10) Pub. No.: US 2025/0045835 A1
(43) Pub. Date: Feb. 6, 2025

(54) ENVIRONMENTAL ATTRIBUTES FOR A CURABLE COMPOSITION

(71) Applicant: BASF SE, Ludwigshafen am Rhein (DE)

(72) Inventors: Bastian GRUMBRECHT, Ludwigshafen am Rhein (DE); Steffen Thomas KLOSTERHALFEN, Ludwigshafen am Rhein (DE); Christian KRUEGER, Ludwigshafen am Rhein (DE); Ana ALBA PEREZ, Ludwigshafen am Rhein (DE); Christopher Alec ANDERLOHR, Ludwigshafen am Rhein (DE); Martin BINDER, Ludwigshafen am Rhein (DE); Alessandro PISTILLO, Ludwigshafen am Rhein (DE); Martin KLATT, Ludwigshafen am Rhein (DE); Kurt WENZL, Ludwigshafen am Rhein (DE)

(21) Appl. No.: 18/719,915

(22) PCT Filed: Dec. 19, 2022
(86) PCT No.: PCT/EP2022/086769

§ 371 Date: Jun. 14, 2024

Related U.S. Application Data
(60) Provisional application No. 63/416,091, filed on Oct. 14, 2022.

(30) Foreign Application Priority Data
Dec. 21, 2021 (EP) 21216268.9
Dec. 21, 2021 (EP) 21216269.7
Dec. 21, 2021 (EP) 21216270.5
Dec. 21, 2021 (EP) 21216271.3
Dec. 21, 2021 (EP) 21216286.1
Dec. 21, 2021 (EP) 21216292.9
Dec. 21, 2021 (EP) 21216326.5
Dec. 21, 2021 (EP) 21216327.3
Dec. 21, 2021 (EP) 21216333.1
Apr. 4, 2022 (EP) 22166573.0
Apr. 12, 2022 (EP) 22167945.9
May 10, 2022 (EP) 22172609.4
May 10, 2022 (EP) 22172611.0
May 10, 2022 (EP) 22172615.1
May 10, 2022 (EP) 22172617.7
May 10, 2022 (EP) 22172619.3
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 5, 2022 (EP) 22211421.7

(51) Int. Cl. G06Q 40/06; G16C 20/90

(52) U.S. Cl. CPC G06Q 40/06; G16C 20/90

(57) ABSTRACT

Disclosed are systems for producing a curable composition associated with a digital asset, methods for producing a curable composition associated with a digital asset, apparatuses for generating a digital asset, computer-implemented methods for generating a chemical passport, computer program elements for generating a digital asset, uses of a curable composition associated with a digital asset, uses of a digital asset, products produced from a curable composition and associated with a digital asset, a digital asset including one or more decentral identifier(s) and data related to the environmental impact data and apparatuses for producing a product associated with the digital asset and methods for producing a product associated with the digital asset.

### TECHNICAL FIELD

The present disclosure relates to systems for producing a curable composition associated with a curable composition passport or digital asset, methods for producing a curable composition associated with a curable composition passport or digital asset, apparatuses for generating a curable composition passport or digital asset, computer-implemented methods for generating a chemical passport, computer program elements for generating a curable composition passport or digital asset, uses of a curable composition associated with a curable composition passport or digital asset, uses of a curable composition passport or digital asset, products produced from a curable composition and associated with a curable composition passport or digital asset, a curable composition, a curable composition passport or digital asset including one or more decentral identifier(s) and data related to the environmental impact datapparatuses for producing a product associated with a curable composition passport or digital asset and methods for producing a product associated with a curable composition passport or digital asset.

### TECHNICAL BACKGROUND

In supply chains the environmental impact of each supply chain participants is of great interest. Specifically in the field of chemistry, curable compositions are employed for a wide range of applications and are supplied to diverse value chains. In such complex systems transparency between value chain participants is hard to achieve.

### SUMMARY OF THE INVENTION

In one aspect disclosed is a system for producing a curable composition associated with a curable composition passport or a digital asset, the system comprising:
a chemical production network configured to produce a curable composition wherein a curable composition is produced from one or more input material(s) through one or more chemical process(s) of the chemical production network, wherein the one or more input material(s) and/or the one or more chemical process(s) are associated with environmental attribute(s);
   a production operating apparatus configured to generate a curable composition passport or digital asset by providing a decentral identifier associated with the produced a curable composition and one or more environmental attribute(s) of the one or more input material(s) and/or the one or more chemical process(s);
   linking the decentral identifier and the environmental attribute(s);

a chemical production network or system configured to provide each produced a curable composition in association with the digital asset.

In another aspect disclosed is a system for producing a curable composition associated with a curable composition passport or digital asset, the system comprising:
a chemical production network configured to produce a curable composition from one or more input material(s) through chemical process(s), wherein the one or more input material(s) and/or the chemical process(s) are associated with environmental attribute(s);
a production operating apparatus configured to generate a curable composition passport or digital asset by providing and/or linking a decentral identifier associated with a curable composition and one or more environmental attribute(s) associated with the one or more input material(s) and/or the chemical process(s).

In another aspect disclosed is a method for producing a curable composition associated with a curable composition passport or digital asset, wherein the method comprises:
producing a curable composition from one or more input material(s) through one or more chemical process(s) of a chemical production network, wherein the one or more input material(s) and/or the one or more chemical process(s) are associated with environmental attribute(s);
generating a curable composition passport or digital asset by
providing a decentral identifier associated with the produced a curable composition and one or more environmental attribute(s) associated with the one or more input material(s) and/or the one or more chemical process(s);
linking the decentral identifier and the one or more environmental attribute(s);
providing the produced a curable composition in association with a curable composition passport or digital asset.

In another aspect disclosed is a method for producing a curable composition associated with a curable composition passport or a digital asset, wherein the method comprises:
producing a curable composition from one or more input material(s) through one or more chemical process(s) of a chemical production network, wherein the one or more input material(s) and/or the one or more chemical process(s) are associated with environmental attribute(s),
generating a curable composition passport or digital asset by providing and/or linking a decentral identifier associated with a curable composition and one or more environmental attribute(s) of the one or more input material(s) and/or the chemical process(s).

In another aspect disclosed is an apparatus for generating a passport or digital asset associated with a curable composition, wherein a curable composition is produced from one or more input material(s) through one or more chemical process(s) of a chemical production network, wherein the one or more input material(s) and/or the one or more chemical process(s) are associated with environmental attribute(s), the apparatus comprising:
providing a decentral identifier associated with the produced a curable composition and one or more environmental attribute(s) of the one or more input material(s) and/or the one or more chemical process(s) used to produce a curable composition;
linking the decentral identifier and the environmental attribute(s);
providing a curable composition passport or digital asset in association with the produced a curable composition e.g. to a decentral network, wherein the environmental attribute(s) associated with a curable composition is made accessible to a user of a curable composition through a curable composition passport or digital asset.

In another aspect disclosed is a method, e.g. A. A computer-implemented method, for generating a curable composition passport or digital asset associated with a curable composition, wherein a curable composition is produced from one or more input material(s) through one or more chemical process(s) of a chemical production network, wherein the one or more input material(s) and/or the one or more chemical process(s) are associated with environmental attribute(s), the method comprising:
providing a decentral identifier associated with the produced a curable composition and one or more environmental attribute(s) of the one or more input material(s) and/or the one or more chemical process(s) used to produce a curable composition;
linking the decentral identifier and the environmental attribute(s);
providing a curable composition passport or digital asset in association with the produced a curable composition e.g. to a decentral network, wherein the environmental attribute(s) associated with a curable composition is made accessible to a user of a curable composition through a curable composition passport or digital asset.

In another aspect disclosed is a computer element, such as a computer readable storage medium, a computer program or a computer program product, comprising instructions, which when executed by a computing node or a computing system, direct the computing node or computing system to carry out the steps of the computer-implemented methods disclosed herein.

In another aspect disclosed is a computer element, such as a computer readable storage medium, a computer program or a computer program product, comprising instructions, which when executed by the apparatuses disclosed herein, direct the apparatuses to carry out steps the apparatuses disclosed herein are configured to execute.

In another aspect disclosed is a curable composition associated with a curable composition passport or a digital asset as produced according to the methods disclosed herein. In another aspect disclosed is a curable composition associated with a curable composition passport or digital asset as produced according to the systems disclosed herein.

In another aspect disclosed is a curable composition associated with a curable composition passport or digital asset, wherein a curable composition is produced from one or more input material(s) through one or more chemical process(s) of a chemical production network, wherein the one or more input material(s) and/or the one or more chemical process(s) are associated with environmental attribute(s), wherein a curable composition passport or digital asset includes a decentral identifier associated with the produced queous polymeric composition and/or formulations using said aqueous polymeric compositions and a link to environmental attribute(s) associated with one or more environmental attribute(s) of one or more input material(s) and/or one or more chemical process(s) used to produce a curable composition.

In another aspect disclosed is a curable composition passport or digital asset as generated according to the methods disclosed herein. In another aspect disclosed is a curable composition passport or digital asset as generated according to the apparatuses disclosed herein.

In another aspect disclosed is a production system for producing a product from a curable composition associated with a curable composition passport or digital asset as provided according to the systems, apparatuses or methods disclosed herein. In another aspect disclosed is a production method for producing a product from a curable composition associated with a curable composition passport or digital asset as provided according to the systems, apparatuses or methods disclosed herein.

In another aspect disclosed is a use of a curable composition associated with a curable composition passport or digital asset as disclosed herein for producing a product from a curable composition associated with a curable composition passport or digital asset.

In another aspect disclosed is a use of a curable composition passport or digital asset as disclosed herein for generating a product passport or digital asset associated with a product produced from a curable composition associated with a curable composition passport or digital asset.

In another aspect disclosed is a method for using the digital asset generated according to the methods disclosed herein in production of a product produced from a curable composition associated with a curable composition passport or digital asset.

In another aspect disclosed is a curable composition associated with a digital asset including a decentral identifier associated with a curable composition and linked to one or more environmental attribute(s) of the one or more input material(s) and/or the one or more chemical process(s) used to produce a curable composition.

In another aspect disclosed is a use of a curable composition associated with a curable composition passport or digital asset for producing a product from a curable composition and associating a curable composition passport or digital asset with the product produced from a curable composition. In another aspect disclosed is a use of a curable composition associated with a curable composition passport or digital asset for producing a product from a curable composition and deriving a product passport or digital asset from a curable composition passport or digital asset. In another aspect disclosed is a method for using a curable composition associated with the digital asset for producing a product from a curable composition as disclosed herein and deriving a digital asset associated with the product from a curable composition passport or a curable composition digital asset.

Any disclosure and embodiments described herein relate to the methods, the systems, chemical products, a curable composition, a curable composition passports, digital assets and the computer elements lined out above and below. Advantageously, the benefits provided by any of the embodiments and examples equally apply to all other embodiments and examples.

### EMBODIMENTS

In the following, embodiments of the present disclosure will be outlined by ways of embodiments and/or example. It is to be understood that the present disclosure is not limited to said embodiments and/or examples.

Determining, generating includes initiating or causing to determine, generate. Providing includes “initiating or causing to access, determine, generate, send or receive”. “Initiating or causing to perform an action” includes any processing signal that triggers a computing node to perform the respective action.

The methods, the systems, the curable compositions, curable composition passports, digital assets and the computer elements disclosed herein provide an efficient, secure and robust way for sharing or exchanging environmental impact data across different participant nodes in value chains. In particular, by providing a curable composition specific data via a curable composition passport or digital asset, environmental impacts can be shared and made transparent from the material to the product produced from such material. A curable composition passport or digital asset enables secure data exchange, since data access can be controlled by a curable composition provider. The exchanged data assets can be specific to a curable composition as produced and tailored to the needs of a curable composition user. This way an improved tracking and tracing of a curable composition can be achieved by securely providing environmental impact data in diverse and highly complex value chains. The environmental impact of a curable composition can hence be tracked leading to simpler, more efficient and sustainable handling of a curable composition by value chain participants.

In an embodiment, a curable composition being associated with the composition passport or digital asset may be selected from compositions comprising at least one compound having at least one functional group selected from the group of (meth)acrylate, isocyanate and epoxy.

A curable composition associated with a curable composition passport or digital asset may be selected from e.g., compositions comprising at least one compound selected from the group of radiation curable compounds, (poly) isocyanate containing compounds or epoxide group containing compounds.

Polyisocyanates for the purposes of the invention are polyisocyanates in their as-synthesized form, and mixtures of these polyisocyanates.

They include the compounds described in more detail below.

The monomeric isocyanates used for preparing the polyisocyanates may be aromatic, aliphatic or cycloaliphatic, preferably aliphatic or cycloaliphatic, as referred to in abbreviated form in this specification as (cyclo) aliphatic; aliphatic isocyanates are particularly preferred.

Aromatic isocyanates are those which comprise at least one aromatic ring system, thus including both purely aromatic and also araliphatic compounds.

Cycloaliphatic isocyanates are those which comprise at least one cycloaliphatic ring system.

Aliphatic isocyanates are those which comprise exclusively linear or branched chains, i.e., acyclic compounds.

The monomeric isocyanates are preferably diisocyanates, which carry precisely two isocyanate groups. They can, however, in principle also be monoisocyanates, having one isocyanate group.

In principle, higher isocyanates having on average more than 2 isocyanate groups are also contemplated. Suitability therefor is possessed for example by triisocyanates such as triisocyanatononane, 2′-isocyanatoethyl (2,6-diisocyanatohexanoate), 2,4,6-triisocyanatotoluene, triphenylmethane triisocyanate or 2,4,4′-tri-isocyanatodiphenyl ether, or the mixtures of diisocyanates, triisocyanates, and higher polyisocyanates that are obtained, for example, by phosgenation of corresponding aniline/formaldehyde condensates and represent methylene-bridged polyphenyl polyisocyanates, more particularly triisocyanatononane and 2′-isocyanatoethyl-(2,6-diisocyanatohexanoate).

The monomeric isocyanates are preferably isocyanates having 4 to 20 C atoms. Examples of typical diisocyanates are aliphatic diisocyanates such as tetramethylene diisocyanate, pentamethylene 1,5-diisocyanate, hexamethylene diisocyanate (1,6 diisocyanatohexane), octamethylene diisocyanate, decamethylene diisocyanate, dodecamethylene diisocyanate, tetradecamethylene diisocyanate, derivatives of lysine diisocyanate, (e.g., methyl 2,6-diisocyanatohexanoate or ethyl 2,6-diisocyanatohexanoate) trimethylhexane diisocyanate or tetramethylhexane diisocyanate, cycloaliphatic diisocyanates such as 1,4-, 1,3- or 1,2-diisocyanatocyclohexane, 4,4′- or 2,4′-di(isocyanatocyclohexyl) methane, 1-isocyanato-3,3,5-trimethyl-5¬(isocyanatomethyl)cyclohexane (isophorone diisocyanate), 1,3- or 1,4-bis-(isocyanatomethyl) cyclohexane or 2,4-, or 2,6-diisocyanato-1 methylcyclohexane, and also 3 (or 4), 8 (or 9) bis(isocyanatomethyl)tricyclo [5.2.1.02,6]decane isomer mixtures, and also aromatic diisocyanates such as tolylene 2,4- or 2,6-diisocyanate and the isomer mixtures thereof, m- or p-xylylene diisocyanate, 2,4′- or 4,4′-diisocyanatodiphenylmethane and the isomer mixtures thereof, phenylene 1,3- or 1,4-diisocyanate, 1-chlorophenylene 2,4-diiso-cyanate, naphthylene 1,5-diisocyanate, diphenylene 4,4′-diisocyanate, 4,4′ diisocyanato-3,3′ dimethylbiphenyl, 3 methyldiphenylmethane 4,4′-diisocyanate, tetramethylxylylene diisocyanate, 1,4 diisocyanatobenzene or diphenyl ether 4,4′ diisocyanate.

Particular preference is given to hexamethylene 1,6-diisocyanate, 1,3-bis(isocyanatomethyl)-cyclohexane, isophorone diisocyanate, and 4,4′- or 2,4′-di(isocyanatocyclohexyl) methane, very particular preference to isophorone diisocyanate and hexamethylene 1,6-diisocyanate, and special preference to hexamethylene 1,6-diisocyanate.

Mixtures of said isocyanates may also be present.

Isophorone diisocyanate is usually in the form of a mixture, specifically a mixture of the cis and trans isomers, generally in a proportion of about 60:40 to 90:10 (w/w), preferably in a proportion of 70:30 to 90:10.

Dicyclohexylmethane 4,4′-diisocyanate may likewise be in the form of a mixture of the different cis and trans isomers.

For the present invention it is possible to use not only those diisocyanates obtained by phosgenating the corresponding amines but also those prepared without the use of phosgene, i.e., by phosgene-free processes. According to EP-A-0 126 299 (U.S. Pat. No. 4,596,678), EP-A-126 300 (U.S. Pat. No. 4,596,679), and EP-A-355 443 (U.S. Pat. No. 5,087,739), for example, (cyclo) aliphatic diisocyanates, such as hexamethylene 1,6-diiso-cyanate (HDI), isomeric aliphatic diisocyanates having 6 carbon atoms in the alkylene radical, 4,4′- or 2,4′-di(isocyanatocyclohexyl) methane, and 1-isocyanato-3 isocyanatomethyl-3,5,5-trimethylcyclohexane (isophorone diisocyanate or IPDI) can be prepared by reacting the (cyclo) aliphatic diamines with, for example, urea and alcohols to give (cyclo) aliphatic biscarbamic esters and subjecting said esters to thermal cleavage into the corresponding diisocyanates and alcohols. The synthesis takes place usually continuously in a circulation process and in the presence, optionally, of N-unsubstituted carbamic esters, dialkyl carbonates, and other by-products recycled from the reaction process. Diisocyanates obtained in this way generally contain a very low or even unmeasurable fraction of chlorinated compounds, which is advantageous, for example, in applications in the electronics industry.

In one embodiment of the present invention the isocyanates used have a hydrolyzable chlorine content of less than 100 ppm, preferably of less than 50 ppm, more preferably less than 30 ppm, especially less than 20 ppm. This can be measured by means, for example, of aSTM specification D4663-98. The amounts of total chlorine are for example below 1000 ppm, preferably below 800 ppm, and more preferably below 500 ppm (determined by argentometric titration after hydrolysis).

It will be appreciated that it is also possible to employ mixtures of those monomeric isocyanates which have been obtained by reacting the (cyclo) aliphatic diamines with, for example, urea and alcohols and cleaving the resulting (cyclo) aliphatic biscarbamic esters, with those diisocyanates which have been obtained by phosgenating the corresponding amines.

The polyisocyanates which can be formed by oligomerizing the monomeric isocyanates are generally characterized as follows:

The average NCO functionality of such compounds is in general at least 1.8 and can be up to 8, preferably 2 to 5, and more preferably 2.4 to 4.

The isocyanate group content after oligomerization, calculated as NCO=42 g/mol, is generally from 5% to 25% by weight unless otherwise specified.

The polyisocyanates are preferably compounds as follows:

1) Polyisocyanates containing isocyanurate groups and derived from aromatic, aliphatic and/or cycloaliphatic diisocyanates. Particular preference is given in this context to the corresponding aliphatic and/or cycloaliphatic isocyanatoisocyanurates and in particular to those based on hexamethylene diisocyanate and isophorone diisocyanate. The isocyanurates present are, in particular, trisisocyanatoalkyl and/or trisisocyanatocycloalkyl isocyanurates, which constitute cyclic trimers of the diisocyanates, or are mixtures with their higher homologs containing more than one isocyanurate ring. The isocyanatoisocyanurates generally have an NCO content of 10% to 30% by weight, in particular 15% to 25% by weight, and an average NCO functionality of 2.6 to 8.

The polyisocyanates containing isocyanurate groups may to a more minor extent also comprise urethane groups and/or allophanate groups, preferably with a bound alcohol content of less than 2% based on the polyisocyanate.

2) Polyisocyanates containing uretdione groups and having aromatically, aliphatically and/or cycloaliphatically attached isocyanate groups, preferably aliphatically and/or cycloaliphatically attached, and in particular those derived from hexamethylene diisocyanate or isophorone diisocyanate. Uretdione diisocyanates are cyclic dimerization products of diisocyanates.

The polyisocyanates containing uretdione groups are frequently obtained as a mixture with other polyisocyanates, more particularly those specified under 1). Polyisocyanates containing uretdione groups typically have functionalities of 2 to 3. This also includes uretdione/isocyanurate mixtures of any desired composition, more particularly having a monomeric uretdione (dimer) content of 1-40%, in particular 3-15, more particularly 5 10%.

For this purpose the diisocyanates can be reacted under reaction conditions under which not only uretdione groups but also the other polyisocyanates are formed, or the uretdione groups are formed first of all and are subsequently reacted to give the other polyisocyanates, or the diisocyanates are first reacted to give the other polyisocyanates, which are subsequently reacted to give products containing uretdione groups.

3) Polyisocyanates containing biuret groups and having aromatically, cyclo-aliphatically or aliphatically attached, preferably cycloaliphatically or aliphatically attached, isocyanate groups, especially tris(6-isocyanatohexyl) biuret or its mixtures with its higher homologs. These polyisocyanates containing biuret groups generally have an NCO content of 18% to 24% by weight and an average NCO functionality of 2.8 to 6.

4) Polyisocyanates containing urethane and/or allophanate groups and having aromatically, aliphatically or cycloaliphatically attached, preferably aliphatically or cycloaliphatically attached, isocyanate groups, such as may be obtained, for example, by reacting excess amounts of diisocyanate, such as of hexamethylene diisocyanate or of isophorone diisocyanate, with mono- or polyhydric alcohols. These polyisocyanates containing urethane and/or allophanate groups generally have an NCO content of 12% to 24% by weight and an average NCO functionality of 2.0 to 4.5. Polyisocyanates of this kind containing urethane and/or allophanate groups may be prepared without catalyst or, preferably, in the presence of catalysts, such as ammonium carboxylates or ammonium hydroxides, for example, or allophanatization catalysts, such as bismuth, cobalt, cesium, Zn(II) or Zr(IV) compounds, for example, in each case in the presence of monohydric, dihydric or polyhydric, preferably monohydric, alcohols.

These polyisocyanates containing urethane groups and/or allophanate groups frequently occur in mixed forms with the polyisocyanates identified under 1).

5) Polyisocyanates comprising oxadiazinetrione groups, derived preferably from hexamethylene diisocyanate or isophorone diisocyanate. Polyisocyanates of this kind comprising oxadiazinetrione groups are accessible from diisocyanate and carbon dioxide.

6) Polyisocyanates comprising iminooxadiazinedione groups, derived preferably from hexamethylene diisocyanate or isophorone diisocyanate. Polyisocyanates of this kind comprising iminooxadiazinedione groups are preparable from diisocyanates by means of specific catalysts.

7) Hyperbranched polyisocyanates, of the kind known for example from DE a1 10013186 or DE-A1 10013187.

8) Polyurethane-polyisocyanate prepolymers, from di- and/or polyisocyanates with alcohols.

9) Polyurea-polyisocyanate prepolymers.

10) The polyisocyanates 1)-9), preferably 1), 3), 4), and 6), can be converted, following their preparation, into polyisocyanates containing biuret groups or urethane/allophanate groups and having aromatically, cycloaliphatically or aliphatically attached, preferably (cyclo) aliphatically attached, isocyanate groups. The formation of biuret groups, for example, is accomplished by addition of water or by reaction with amines. The formation of urethane and/or allophanate groups is accomplished by reaction with monohydric, dihydric or polyhydric, preferably monohydric, alcohols, in the presence if desired of suitable catalysts. These polyisocyanates containing biuret or urethane/allophanate groups generally have an NCO content of 10% to 25% by weight and an average NCO functionality of 3 to 8.

11) Hydrophilically modified polyisocyanates, i.e., polyisocyanates which as well as the groups described under 1-10 also comprise groups which result formally from addition of molecules containing NCO-reactive groups and hydrophilizing groups to the isocyanate groups of the above molecules. The latter groups are nonionic groups such as alkylpolyethylene oxide and/or ionic groups derived from phosphoric acid, phosphonic acid, sulfuric acid or sulfonic acid, and/or their salts.

12) Modified polyisocyanates for dual cure applications, i.e., polyisocyanates which as well as the groups described under 1-11 also comprise groups resulting formally from addition of molecules containing NCO-reactive groups and UV crosslinkable or actinic-radiation-crosslinkable groups to the isocyanate groups of the above molecules. These molecules are, for example, hydroxyalkyl (meth)acrylates and other hydroxyvinyl compounds.

Epoxide group containing compounds may be epoxy resins.

Epoxy resins are polymers carrying epoxy groups. Epoxy polymers can be obtained by reaction of polyols with epichlorohydrin followed by dehydrohalogenation. Examples of polyols are bisphenol a and bisphenol F as well as novolak resins, which are polymers formed by reaction of phenol with formaldehyde.

Epoxy resins are for example described in H. Q. Pham, M. J. Marks “Epoxy Resins”, DOI: 10.1002/14356007.a09_547.pub2 in “Ullmann's Encyclopedia of Industrial Chemistry-7th ed.”, Whurr Publishers, in Z. W. Wicks, F. N. Jones, S. P. Pappas “Organic Coatings: Science and Technology”, vol. I, chapter XI “Epoxy and Phenolic Resins”, John Wiley & Sons, and in G. Bianchini, R. Dyer, a. Fream, P. Heffer, S. P. McOwan, P. Oldring, I. Royston, N. Tuck, “Waterborne & Solvent Based Epoxies and their End User applications” in “Waterborne & Solvent Based Surface Coatings Resins and Their applications”, Vol. II, SITA Technology.

The radiation curable compound may be a polymer carrying ethylenically unsaturated groups. The polymer may be selected from the group consisting of polyurethanes, acrylic polymers, hybrids of polyurethane and acrylic polymer, polyesters, polyethers, polycarbonates, epoxy resins, alkyd resins, polyolefins and polyvinylacetate, as well as acryated or methacrylated derivatives thereof.

The radiation-curable compound preferably comprises at least one binder selected from the group consisting of
i) polymers having ethylenically unsaturated groups and having an average molar mass Mn of more than 2000 g/mol
ii) mixtures of i) with ethylenically unsaturated, low molecular weight compounds differing from i) and having a molar mass of less than 2000 g/mol
iii) mixtures of saturated thermoplastic polymers with ethylenically unsaturated compounds.

### Re i)

Suitable polymers are, for example, polymers of ethylenically unsaturated compounds but also polyesters, polyethers, polycarbonates, polyepoxides or polyurethanes having a molar mass of more than 2000 g/mol.

For example, unsaturated polyester resins which substantially comprise polyols, in particular diols, and polycarboxylic acid, in particular dicarboxylic acid, are suitable, one of the esterification components comprising a copolymerizable, ethylenically unsaturated group. For example, this is maleic acid, fumaric acid or maleic anhydride.

Polymers of ethylenically unsaturated compounds as obtained in particular by free radical polymerization are preferred.

The polymers obtained by free radical polymerization are in particular polymers which are composed of more than 40% by weight, particularly preferably of more than 60% by weight, of acrylate monomers, in particular C1-C8-alkyl (meth)acrylates, preferably C1-C4-alkyl (meth)acrylates, particularly preferably methyl (meth)acrylate, ethyl (meth)acrylate or n-butyl (meth)acrylate.

As ethylenically unsaturated groups, the polymers comprise, for example, vinyl ether and/or in particular (meth)acrylate groups. These may be bonded to the polymer, for example, by reaction of (meth)acrylic acid with epoxide groups in the polymer (for example by the concomitant use of glycidyl (meth)acrylate as a comonomer).

Epoxide (meth)acrylates are obtainable by reacting epoxides with (meth)acrylic acid. Suitable epoxides are, for example, epoxidized olefins, aromatic glycidyl ethers or aliphatic glycidyl ethers, preferably those of aromatic or aliphatic glycidyl ethers.

Epoxidized olefins may be, for example, ethylene oxide, propylene oxide, isobutylene oxide, 1-butene oxide, 2-butene oxide, vinyloxirane, styrene oxide or epichlorohydrin, preferably ethylene oxide, propylene oxide, isobutylene oxide, vinyloxirane, styrene oxide or epichlorohydrin, particularly preferably ethylene oxide, propylene oxide or epichlorohydrin and very particularly preferably ethylene oxide and epichlorohydrin.

Aromatic glycidyl ethers are, for example, bisphenol A diglycidyl ether, bisphenol F diglycidyl ether, bisphenol B diglycidyl ether, bisphenol S diglycidyl ether, hydroquinone diglycidyl ether, alkylation products of phenol/dicyclopentadiene, e.g. 2,5-bis [(2,3-epoxypropoxy)phenyl] octahydro-4,7-methano-5H-indene) (CAS No. [13446-85-0]), tris [4-(2,3-epoxypropoxy)phenyl] methane isomers) (CAS No. [66072-39-7]), phenol-based epoxy novolaks (CAS No. [9003-35-4]) and cresol-based epoxy novolaks (CAS No. [37382-79-9]).

Aliphatic glycidyl ethers are, for example, 1,4-butanediol diglycidyl ether, 1,6-hexanediol diglycidyl ether, trimethylolpropane triglycidyl ether, pentaerythrityl tetraglycidyl ether, 1,1,2,2-tetrakis [4-(2,3-epoxypropoxy)phenyl] ethane (CAS No. [27043-37-4]), diglycidyl ethers of polypropylene glycol (-bis(2,3-epoxypropoxy) poly(oxypropylene) (CAS No. [16096-30-3]) and of hydrogenated bisphenol A (2,2-bis [4-(2,3-epoxypropoxy)cyclohexyl] propane (CAS No. [13410-58-7]).

The epoxide (meth)acrylates and epoxide vinyl ethers preferably have a number average molecular weight Mn of from 2000 to 20 000, particularly preferably from 2000 to 10 000, g/mol and very particularly preferably from 2000 to 3000 g/mol; the content of (meth)acrylate and vinyl ether groups is preferably from 1 to 5, particularly preferably from 2 to 4, per 1000 g of epoxide (meth)acrylate or vinyl ether epoxide (determined by gel permeation chromatography using polystyrene as a standard and tetrahydrofuran as an eluent).

Polyurethanes are likewise preferred. These likewise preferably comprise, as unsaturated groups, (meth)acrylate groups which are bonded to the polyurethane, for example, by reaction of hydroxyalkyl (meth)acrylates with isocyanate groups.

Such urethane (meth)acrylates are obtainable, for example, by reacting polyisocyanates with hydroxyalkyl (meth)acrylates or hydroxyalkyl vinyl ethers and, if appropriate, chain extenders, such as diols, polyols, diamines, polyamines or dithiols or polythiols. Urethane (meth)acrylates dispersible in water without addition of emulsifiers additionally contain ionic and/or nonionic hydrophilic groups, which are introduced into the urethane, for example, by components such as hydroxcarboxylic acids.

The polyurethanes which can be used as binders substantially comprise as components:
(a) at least one organic aliphatic, aromatic or cycloaliphatic di- or polyisocyanate,
(b) at least one compound having at least one group reactive toward isocyanate and at least one unsaturated group capable of free radical polymerization and
(c) if appropriate, at least one compound having at least two groups reactive toward isocyanate.

For example, aliphatic, aromatic and cycloaliphatic di- and polyisocyanates having an NCO functionality of at least 1.8, preferably from 1.8 to 5 and particularly preferably from 2 to 4, and the isocyanurates, biurets, allophanates and uretdiones thereof, are suitable as component (a).

The diisocyanates are preferably isocyanates having 4 to 20 carbon atoms. Examples of conventional diisocyanates are aliphatic diisocyanates, such as tetramethylene diisocyanate, hexamethylene diisocyanate (1,6-diisocyanatohexane), octamethylene diisocyanate, decamethylene diisocyanate, dodecamethylene diisocyanate, tetradecamethylene diisocyanate, derivatives of lysine diisocyanate, tetramethylxylylene diisocyanate, trimethylhexane diisocyanate or tetramethylhexane diisocyanate, cycloaliphatic diisocyanates, such as 1,4-, 1,3- or 1,2-diisocyanatocyclohexane, 4,4′- or 2,4′-di(isocyanatocyclohexyl) methane, 1-isocyanato-3,3,5-trimethyl-5-(isocyanatomethyl)cyclohexane (isophorone diisocyanate), 1,3- or 1,4-bis(isocyanatomethyl) cyclohexane or 2,4- or 2,6-diisocyanato-1-methylcyclohexane, and aromatic diisocyanates, such as toluene 2,4- or 2,6-diisocyanate and the isomer mixtures thereof, m- or p-xylylene diisocyanate, 2,4′- or 4,4′-diisocyanatodiphenylmethane and the isomer mixtures thereof, phenylene 1,3- or 1,4-diisocyanate, 1-chlorophenylene 2,4-diisocyanate, naphthylene 1,5-diisocyanate, biphenylene 4,4′-diisocyanate, 4,4′-diisocyanato-3,3′-dimethylbiphenyl, 3-methyldiphenylmethane 4,4′-diisocyanate, tetramethylxylylene diisocyanate, 1,4-diisocyanatobenzene or 4,4′-diisocyanatodiphenyl ether.

Mixtures of said diisocyanates may also be present.

Hexamethylene diisocyanate, 1,3-bis(isocyanatomethyl)cyclohexane, isophorone diisocyanate and di(isocyanatocyclohexyl) methane are preferred.

Suitable polyisocyanates are polyisocyanates having isocyanurate groups, uretdione diisocyanates and polyisocyanates having biuret groups, polyisocyanates having urethane or allophanate groups, polyisocyanates comprising oxadiazinetrione groups, uretonimine-modified polyisocyanates of linear or branched C4-C20-alkylene diisocyanates, cycloaliphatic diisocyanates having in total 6 to 20 carbon atoms or aromatic diisocyanates having in total 8 to 20 carbon atoms or mixtures thereof.

The di- and polyisocyanates which can be used preferably contain from 10 to 60% by weight, based on the di- and polyisocyanate (mixture), preferably from 15 to 60% by weight and particularly preferably from 20 to 55% by weight, of isocyanate groups (calculated as NCO, molecular weight=42).

Aliphatic or cycloaliphatic di- and polyisocyanates, for example the abovementioned aliphatic or cycloaliphatic diisocyanates, or mixtures thereof, are preferred.

The following are furthermore preferred:

1) Polyisocyanates having isocyanurate groups and obtained from aromatic, aliphatic and/or cycloaliphatic diisocyanates. Particularly preferred here are the corresponding aliphatic and/or cycloaliphatic isocyanatoisocyanurates and in particular those based on hexamethylene diisocyanate and isophorone diisocyanate. The isocyanurates present are in particular trisisocyanatoalkyl or trisisocyanatocycloalkyl isocyanurates, which are cyclic trimers of the diisocyanates, or are mixtures with their higher homologs having more than one isocyanurate ring. The isocyanatoisocyanurates generally have an NCO content of from 10 to 30% by weight, in particular from 15 to 25% by weight, and an average NCO functionality of from 3 to 4.5.

2) Uretdione diisocyanates having aromatically, aliphatically and/or cycloaliphatically bonded isocyanate groups, preferably having aliphatically and/or cycloaliphatically bonded groups and in particular those derived from hexamethylene diisocyanate or isophorone diisocyanate. Uretdione diisocyanates are cyclic dimerization products of diisocyanates.

The uretdione diisocyanates can be used in the formulations as a sole component or as a mixture with other polyisocyanates, in particular those mentioned under 1).

3) Polyisocyanates having biuret groups and having aromatically, cycloaliphatically or aliphatically bonded, preferably cycloaliphatically or aliphatically bonded, isocyanate groups, in particular tris(6-isocyanatohexyl) biuret or mixtures thereof with its higher homologs. These polyisocyanates having biuret groups generally have an NCO content of from 18 to 22% by weight and an average NCO functionality of from 3 to 4.5.

4) Polyisocyanates having urethane and/or allophanate groups and having aromatically, aliphatically or cycloaliphatically bonded, preferably aliphatically or cycloaliphatically bonded, isocyanate groups, as can be obtained, for example, by reaction of excess amounts of hexamethylene diisocyanate or of isophorone diisocyanate with polyhydric alcohols, such as, for example, trimethylolpropane, neopentylglycol, pentaerythritol, 1,4-butanediol, 1,6-hexanediol, 1,3-propanediol, ethylene glycol, diethylene glycol, glycerol, 1,2-dihydroxypropane or mixtures thereof. These polyisocyanates having urethane and/or allophanate groups generally have an NCO content of from 12 to 20% by weight and an average NCO functionality of from 2.5 to 3.

5) Polyisocyanates comprising oxadiazinetrione groups, preferably derived from hexamethylene diisocyanate or isophorone diisocyanate. Such polyisocyanates comprising oxadiazinetrione groups can be prepared from diisocyanate and carbon dioxide.

6) Uretonimine-modified polyisocyanates.

The polyisocyanates 1) to 6) can be used as a mixture, if appropriate also as a mixture with diisocyanates.

Compounds suitable as component (b) are those which carry at least one group reactive toward isocyanate and at least one group capable of free radical polymerization.

Groups reactive toward isocyanates may be, for example, —OH, SH, NH2 and NHR1, where R1 is hydrogen or an alkyl group comprising 1 to 4 carbon atoms, such as, for example, methyl, ethyl, n-propyl, isopropyl, n-butyl, isobutyl, sec-butyl or tert-butyl.

Components (b) may be, for example, monoesters of □,□-unsaturated carboxylic acids, such as acrylic acid, methacrylic acid, crotonic acid, itaconic acid, fumaric acid, maleic acid, acrylamidoglycolic acid or methacrylamidoglycolic acid, or vinyl ethers with di- or polyols, which preferably have 2 to 20 carbon atoms and at least two hydroxyl groups, such as ethylene glycol, diethylene glycol, triethylene glycol, 1,2-propylene glycol, 1,3-propylene glycol, 1,1-dimethyl-1,2-ethanediol, dipropylene glycol, triethylene glycol, tetraethylene glycol, pentaethylene glycol, tripropylene glycol, 1,4-butanediol, 1,5-pentanediol, neopentylglycol, 1,6-hexanediol, 2-methyl-1,5-pentanediol, 2-ethyl-1,4-butanediol, 1,4-dimethylolcyclohexane, 2,2-bis(4-hydroxycyclohexyl) propane, glycerol, trimethylolethane, trimethylolpropane, trimethylolbutane, pentaerythritol, ditrimethylolpropane, erythritol, sorbitol, poly-THF having a molecular weight of from 162 to 2000, poly-1,3-propanediol having a molecular weight of from 134 to 400 or polyethylene glycol having a molecular weight of from 238 to 458. It is furthermore possible to use esters or amides of (meth)acrylic acid with amino alcohols, e.g. 2-aminoethanol, 2-(methylamino) ethanol, 3-amino-1-propanol, 1-amino-2-propanol or 2-(2-aminoethoxy) ethanol, 2-mercaptoethanol or polyaminoalkanes, such as ethylenediamine or diethylenetriamine, or vinylacetic acid.

Unsaturated polyetherols or polyesterols or polyacrylatepolyols having an average OH functionality of from 2 to 10 are furthermore suitable.

Examples of amides of ethylenically unsaturated carboxylic acids with amino alcohols are hydroxyalkyl (meth)acrylamides, such as N-hydroxymethylacrylamide, N hydroxymethylmethacrylamide, N-hydroxyethylacrylamide, N hydroxyethyl-methacrylamide, 5-hydroxy-3-oxapentyl (meth)acrylamide, N hydroxyalkylcrotonamides, such as N-hydroxymethylcrotonamide or N hydroxyalkylmaleimides, such as N hydroxyethylmaleimide.

2-Hydroxyethyl (meth)acrylate, 2- or 3-hydroxypropyl (meth)acrylate, 1,4-butanediol mono(meth)acrylate, neopentylglycol mono(meth)acrylate, 1,5-pentanediol mono(meth)acrylate, 1,6-hexanediol mono(meth)acrylate, glyceryl mono- and di(meth)acrylate, trimethylolpropane mono- and di(meth)acrylate, pentaerythrityl mono, di- and tri(meth)acrylate, and 4-hydroxybutyl vinyl ether, 2-aminoethyl (meth)acrylate, 2-aminopropyl (meth)acrylate, 3-aminopropyl (meth)acrylate, 4 aminobutyl (meth)acrylate, 6-aminohexyl (meth)acrylate, 2-thioethyl (meth)acrylate, 2-aminoethyl (meth)acrylamide, 2-aminopropyl (meth)acrylamide, 3-aminopropyl-(meth) acrylamide, 2-hydroxyethyl (meth)acrylamide, 2-hydroxypropyl (meth)acrylamide or 3-hydroxypropyl (meth)acrylamide are preferably used. 2-Hydroxyethyl acrylate, 2 hydroxyethyl methacrylate, 2- or 3-hydroxypropyl acrylate, 1,4-butanediol monoacrylate and 3-(acryloyloxy)-2-hydroxypropyl methacrylate are particularly preferred.

Compounds which are suitable as component (c) are those which have at least two groups reactive toward isocyanate, for example —OH, SH, —NH2 or NHR2, where R2 therein, independently of one another, may be hydrogen, methyl, ethyl, isopropyl, n propyl, n-butyl, isobutyl, sec-butyl or tert-butyl.

These are preferably diols or polyols, such as hydrocarbondiols having 2 to 20 carbon atoms, e.g. ethylene glycol, 1,2-propanediol, 1,3-propanediol, 1,1-dimethylethane-1,2-diol, 1,6-hexanediol, 1,10-decanediol, bis-(4-hydroxycyclohexane) isopropylidene, tetramethylcyclobutanediol, 1,2-, 1,3- or 1,4-cyclohexanediol, cyclooctanediol, norbornanediol, pinanediol, decalindiol, etc., esters thereof with short-chain dicarboxylic acids, such as adipic acid or cyclohexanedicarboxylic acid, carbonates thereof, prepared by reaction of the diols with phosgene or by transesterification with dialkyl or diaryl carbonates, or aliphatic diamines, such as methyleneand isopropylidenebis(cyclohexylamine), piperazine, 1,2-, 1,3- or 1,4-diaminocyclohexane, 1,2-, 1,3- or 1,4-cyclohexanebis(methylamine), etc., dithiols or polyfunctional alcohols, secondary or primary amino alcohols, such as ethanolamine, diethanolamine, monopropanolamine, dipropanolamine, etc., or thioalcohols, such as thioethylene glycol.

Diethylene glycol, triethylene glycol, dipropylene glycol, tripropylene glycol, neopentylglycol, pentaerythritol, 1,2- and 1,4-butanediol, 1,5-pentanediol, 2-methyl-1,5-pentanediol, 2-ethyl-1,4-butanediol, 1,2-, 1,3- and 1,4-dimethylolcyclohexane, 2,2-bis(4-hydroxycyclohexyl) propane, glycerol, trimethylolethane, trimethylolpropane, trimethylolbutane, dipentaerythritol, ditrimethylolpropane, erythritol and sorbitol, 2-aminoethanol, 3-amino-1-propanol, 1-amino-2-propanol or 2-(2-aminoethoxy) ethanol, bisphenol A or butanetriol are furthermore conceivable.

Unsaturated polyetherols or polyesterols or polyacrylatepolyols having an average OH functionality of 2 to 10, and polyamines, such as, for example, polyethylenimine, or polymers of, for example, poly-N-vinylformamide which comprise free amino groups, are furthermore suitable.

The cycloaliphatic diols, such as, for example, bis-(4-hydroxycyclohexane)-isopropylidene, tetramethylcyclobutanediol, 1,2-, 1,3- or 1,4-cyclohexanediol, cyclooctanediol or norbornanediol, are particularly suitable here.

The polyurethanes which can be used according to the invention are obtained by reacting the components (a), (b) and (c) with one another.

The molar composition (a): (b): (c) per 3 mol of reactive isocyanate groups in (a) is as a rule as follows:
(b) 1.5-3.0, preferably 1.5-2.5, particularly preferably 1.5-2.0 and in particular 1.6-1.8 mol of groups reactive toward isocyanate and
(c) 0-1.5, preferably 0.5-1.5, particularly preferably 0.7-1.5 and in particular 0.8-1.5 mol of groups reactive toward isocyanate.

With the use of the polyurethanes in aqueous systems, preferably substantially all isocyanate groups present have reacted.

The formation of the adduct from the compound containing isocyanate groups and the compound which comprises groups reactive toward isocyanate groups is effected, as a rule, by mixing the components in any desired sequence, if appropriate at elevated temperature.

Preferably, the compound which comprises groups reactive toward isocyanate groups is added to the compound containing isocyanate groups, preferably in a plurality of steps.

Particularly preferably, the compound containing isocyanate groups is initially taken and the compounds which comprise groups reactive toward isocyanate are added. In particular, the compound (a) containing isocyanate groups is initially taken and then (b) is added. If appropriate, desired further components can subsequently be added.

As a rule, the reaction is carried out at temperatures between 5 and 100° C., preferably between 2° and 90° C. and particularly preferably between 4° and 80° C. and in particular between 6° and 80° C.

The procedure is preferably carried out under anhydrous conditions.

Here, anhydrous means that the water content in the reaction system is not more than 5% by weight, preferably not more than 3% by weight and particularly preferably not more than 1% by weight.

In order to suppress polymerization of the polymerizable double bonds, the procedure is preferably carried out under an oxygen-containing gas, particularly preferably air or air-nitrogen mixtures.

Air or a mixture of oxygen or air and a gas which is inert under the conditions of use can preferably be used as the oxygen-containing gas. Nitrogen, helium, argon, carbon monoxide, carbon dioxide, steam, lower hydrocarbons or mixtures thereof can be used as the inert gas.

The oxygen content of the oxygen-containing gas may be, for example, from 0.1 to 22% by volume, preferably from 0.5 to 20, particularly preferably from 1 to 15, very particularly preferably from 2 to 10 and in particular from 4 to 10% by volume. If desired, higher oxygen contents can of course also be used.

The reaction can also be carried out in the presence of an inert solvent, e.g. acetone, isobutyl methyl ketone, toluene, xylene, butyl acetate or ethoxyethyl acetate. However, the reaction is preferably carried out in the absence of a solvent.

The urethane (meth)acrylates preferably have a number average molecular weight Mn from 1000 to 20 000, in particular from 1000 to 10 000, particularly preferably from 1000 to 4000, g/mol (determined by gel permeation chromatography using tetrahydrofuran and polystyrene as standard).

The urethane (meth)acrylates preferably contain from 1 to 5, particularly preferably from 2 to 4, mol of (meth)acrylate groups per 1000 g of urethane (meth)acrylate.

The urethane vinyl ethers preferably contain from 1 to 5, particularly preferably from 2 to 4, mol of vinyl ether groups per 1000 g of urethane vinyl ether.

In a preferred embodiment of this invention, the urethane (meth)acrylates or urethane vinyl ethers, preferably urethane acrylates, comprise at least one cycloaliphatic isocyanate, i.e. a compound in which at least one isocyanate group is bonded to a cycloaliphatic, as a component, particularly preferably IPDI.

In a further preferred embodiment, compounds used are those as described in WO 00/39183, page 4, line 3 to page 10, line 19, the disclosure of which is hereby incorporated by reference. Particularly preferred among these are those compounds which have, as components, at least one (cyclo) aliphatic isocyanate having allophanate groups and at least one hydroxyalkyl (meth)acrylate, very particularly preferably product No. 1 to 9 in table 1 on page 24 of WO 00/39183.

Other suitable radiation-curable compounds are carbonate (meth)acrylates which comprise on average preferably from 1 to 5, in particular from 2 to 4, particularly preferably 2 or 3, (meth)acrylate groups and very particularly preferably 2 (meth)acrylate groups.

The number average molecular weight Mn of the carbonate (meth)acrylates is preferably from 2000 to 3000 g/mol (determined by gel permeation chromatography using polystyrene as a standard and tetrahydrofuran as a solvent).

The carbonate (meth)acrylates are obtainable in a simple manner by transesterification of carbonic esters with polyhydric, preferably dihydric, alcohols (diols, e.g. hexanediol) and subsequent esterification of the free OH groups with (meth)acrylic acid, or transesterification with (meth)acrylic esters, as described, for example, in EP A 92 269. They are also obtainable by reacting phosgene, urea derivatives with polyhydric, e.g. dihydric, alcohols.

In an analogous manner, vinyl ether carbonates are also obtainable by reacting a hydroxyalkyl vinyl ether with carbonic esters and, if appropriate, dihydric alcohols.

(Meth)acrylates or vinyl ethers of polycarbonatepolyols, such as reaction product of one of said di- or polyols and a carbonic ester or else a hydroxyl-containing (meth)acrylate or vinyl ether, are also conceivable.

Suitable carbonic esters are, for example, ethylene or 1,2- or 1,3-propylene carbonate or dimethyl, diethyl or dibutyl carbonate.

Suitable hydroxyl-containing (meth)acrylates are, for example, 2-hydroxyethyl (meth)acrylate, 2- or 3-hydroxypropyl (meth)acrylate, 1,4-butanediol mono(meth)acrylate, neopentylglycol mono(meth)acrylate, glyceryl mono- and di(meth)acrylate, trimethylolpropane mono- and di(meth)acrylate and pentaerythrityl mono-, di- and tri(meth)acrylate.

Suitable hydroxyl-containing vinyl ethers are, for example, 2-hydroxyethyl vinyl ether and 4-hydroxybutyl vinyl ether.

Particularly preferred carbonate (meth)acrylates are those of the formula:

where R is H or CH3, X is a C2-C18-alkylene group and n is an integer from 1 to 5, preferably from 1 to 3.

R is preferably H and X is preferably C2-to C10-alkylene, for example 1,2-ethylene, 1,2-propylene, 1,3-propylene, 1,4-butylene or 1,6-hexylene, particularly preferably C4-to C8-alkylene. Very particularly preferably, X is C6-alkylene.

They are preferably aliphatic carbonate (meth)acrylates.

The polymers i) as such are thermoplastically processible before the UV curing.

### Re ii)

The unsaturated polymers i) can be used as mixtures with ethylenically unsaturated, low molecular weight compounds.

In this context, low molecular weight compounds are understood as meaning compounds having a number average molecular weight of less than 2000 g/mol (determined by gel permeation chromatography using polystyrene as a standard).

These may be, for example, those compounds mentioned under i) which have a molar mass of less than 2000 g/mol, for example epoxide (meth)acrylates having a molar mass of 340, preferably 500 and particularly preferably 750 to less than 2000 g/mol, urethane (meth)acrylates having a molar mass of 300, preferably 500 and particularly preferably 750 to less than 2000 g/mol or carbonate (meth)acrylates having a molar mass of 170, preferably 250 and particularly preferably 500 to less than 2000 g/mol.

Furthermore suitable are, for example, compounds capable of free radical polymerization and having only one ethylenically unsaturated, copolymerizable group.

Examples are C1-C20-alkyl (meth)acrylates, vinylaromatics having up to 20 carbon atoms, vinyl esters of carboxylic acids comprising up to 20 carbon atoms, ethylenically unsaturated nitriles, vinyl ethers of alcohols comprising 1 to 10 carbon atoms and aliphatic hydrocarbons having 2 to 20, preferably 2 to 8, carbon atoms and 1 or 2 double bonds.

Preferred alkyl (meth)acrylates are those having a C1-C10-alkyl radical, such as methyl methacrylate, methyl acrylate, n-butyl acrylate, ethyl acrylate and 2-ethylhexyl acrylate.

Mixtures of the alkyl (meth)acrylates are also particularly suitable.

Vinyl esters of carboxylic acids having 1 to 20 carbon atoms are, for example, vinyl laurate, vinyl stearate, vinyl propionate and vinyl acetate.

Suitable vinylaromatic compounds are, for example, vinyltoluene, a-butylstyrene, 4-nbutylstyrene, 4-n-decylstyrene and preferably styrene.

Examples of nitriles are acrylonitrile and methacrylonitrile.

Suitable vinyl ethers are, for example, vinyl methyl ether, vinyl isobutyl ether, vinyl hexyl ether and vinyl octyl ether.

Butadiene, isoprene and ethylene, propylene and isobutylene may be mentioned as nonaromatic hydrocarbons having 2 to 20, preferably 2 to 8, carbon atoms and one or two olefinic double bonds.

Compounds capable of free radical polymerization and having a plurality of ethylenically unsaturated groups are preferred.

These are in particular (meth)acrylate compounds, the acrylate compounds, i.e. the derivatives of acrylic acid, being preferred in each case.

Preferred (meth)acrylate compounds contain 2 to 20, preferably 2 to 10 and very particularly preferably 2 to 6 copolymerizable, ethylenically unsaturated double bonds.

(Meth)acrylic esters and in particular acrylic esters of polyfunctional alcohols, in particular those which comprise no further functional groups or at least ether groups apart from the hydroxyl groups may be mentioned as (meth)acrylate compounds. Examples of such alcohols are, for example, bifunctional alcohols, such as ethylene glycol, propylene glycol and the members thereof having a higher degree of condensation, for example such as diethylene glycol, triethylene glycol, dipropylene glycol, tripropylene glycol, etc., butanediol, pentanediol, hexanediol, neopentylglycol, alkoxylated phenolic compounds, such as ethoxylated or propoxylated bisphenols, cyclohexanedimethanol, trifunctional and higher-functional alcohols, such as glycerol, trimethylolpropane, trimethylolethane, neopentylglycol, pentaerythritol, glycerol, ditrimethylolpropane, dipentaerythritol, sorbitol, mannitol, diglycerol, 1,2-propanediol, ethylene glycol, 2,2-dimethyl-1,2-ethanediol, neopentylglycol, 1,3-propanediol, 1,2-butanediol, 1,4-butanediol, butanetriol, sorbitol, mannitol and the corresponding alkoxylated, in particular ethoxylated and propoxylated, alcohols.

The alkoxylation products are obtainable in a known manner by reaction of the above alcohols with alkylene oxides, for example ethylene oxide, propylene oxide, butylene oxide, isobutylene oxide and vinyloxirane, in any desired sequence or as a mixture, preferably ethylene oxide and/or propylene oxide and particularly preferably ethylene oxide. The degree of alkoxylation per hydroxyl group is preferably from 0 to 10, i.e. 1 mol of hydroxyl can preferably be alkoxylated with up to 10 mol of alkylene oxides.

Polyetheralcohols containing vinyl ether groups are obtained, for example, in a corresponding manner by reaction of hydroxyalkyl vinyl ethers with alkylene oxides.

Polyetheralcohols containing (meth)acrylic acid groups can be obtained, for example, by transesterification of (meth)acrylic esters with the polyetheralcohols, by esterification of the polyetheralcohols with (meth)acrylic acid or by use of hydroxyl-containing (meth)acrylates as described above under (b).

Preferred polyetheralcohols are polyethylene glycols having a molar mass between 106 and 2000, preferably between 106 and 898, particularly preferably between 238 and 678.

Poly-THF having a molar mass between 162 and 2000 and poly-1,3-propanediol having a molar mass between 134 and 1178 can furthermore be used as polyetheralcohols.

Polyester (meth)acrylates may furthermore be mentioned as (meth)acrylate compounds, these being the (meth)acrylic esters of polyesterols.

Polyesterpolyols are known, for example, from Ullmanns Enzyklopädie der technischen Chemie, 4th Edition, Volume 19, pages 62 to 65. Polyesterpolyols which are obtained by reacting dihydric alcohols with dibasic carboxylic acids are preferably used. Instead of the free polycarboxylic acids, it is also possible to use the corresponding polycarboxylic anhydrides or corresponding polycarboxylic esters of lower alcohols or mixtures thereof for the preparation of the polyesterpolyols. The polycarboxylic acids may be aliphatic, cycloaliphatic, araliphatic, aromatic, heterocyclic and, if appropriate, may be, for example, substituted by halogen atoms and/or unsaturated. The following may be mentioned as examples of these:

oxalic acid, maleic acid, fumaric acid, succinic acid, glutaric acid, adipic acid, sebacic acid, dodecanedioic acid, o-phthalic acid, isophthalic acid, terephthalic acid, trimellitic acid, azelaic acid, 1,4-cyclohexanedicarboxylic acid or tetrahydrophthalic acid, suberic acid, azelaic acid, phthalic anhydride, tetrahydrophthalic anhydride, hexahydrophthalic anhydride, tetrachlorophthalic anhydride, endomethylenetetrahydrophthalic anhydride, glutaric anhydride, maleic anhydride, dimeric fatty acids, the isomers and hydrogenation products thereof and esterifiable derivatives, such as anhydrides or dialkyl esters, for example C1-C4-alkyl esters, preferably methyl, ethyl or n-butyl esters, of said acids. Dicarboxylic acids of the general formula HOOC—(CH2)y-COOH, where y is a number from 1 to 20, preferably an even number from 2 to 20, are preferred, succinic acid, adipic acid, sebacic acid and dodecanedicarboxylic acid being particularly preferred.

Suitable polyhydric alcohols for the preparation of the polyesterols are 1,2-propanediol, ethylene glycol, 2,2-dimethyl-1,2-ethanediol, 1,3-propanediol, 1,2-butanediol, 1,3-butanediol, 1,4-butanediol, 3-methylpentane-1,5-diol, 2-ethylhexane-1,3-diol, 2,4-diethyloctane-1,3-diol, 1,6-hexanediol, poly-THF having a molar mass between 162 and 2000, poly-1,3-propanediol having a molar mass between 134 and 1178, poly-1,2-propanediol having a molar mass between 134 and 898, polyethylene glycol having a molar mass between 106 and 458, neopentylglycol, neopentylglycol hydroxypivalate, 2-ethyl-1,3-propanediol, 2-methyl-1,3-propanediol, 2,2-bis(4-hydroxycyclohexyl) propane, 1,1-, 1,2-, 1,3- and 1,4-cyclohexanedimethanol, 1,2-, 1,3- or 1,4-cyclohexanediol, trimethlyolbutane, trimethylolpropane, trimethylolethane, neopentylglycol, pentaerythritol, glycerol, ditrimethylolpropane, dipentaerythritol, sorbitol, mannitol, diglycerol, threitol, erythritol, adonitol (ribitol), arabitol (lyxitol), xylitol, dulcitol (galactitol), maltitol or isomaltitol.

Alcohols of the general formula HO—(CH2)x-OH, where x is a number from 1 to 20, preferably an even number from 2 to 20, are preferred. Ethylene glycol, butane-1,4-diol, hexane-1,6-diol, octane-1,8-diol and dodecane-1,12-diol are preferred. Neopentylglycol is furthermore preferred.

Polycarbonatediols, as can be obtained, for example, by reacting phosgene with an excess of the low molecular weight alcohols mentioned as components for the polyesterpolyols are furthermore suitable.

Lactone-based polyesterdiols are also suitable, these being homo- or copolymers of lactones, preferably adducts of lactones with suitable difunctional initiator molecules, which adducts have terminal hydroxyl groups. Preferred lactones are those which are derived from compounds of the general formula HO—(CH2)z-COOH, where z is a number from 1 to 20 and an H atom of a methylene unit may also be substituted by a C1-to C4-alkyl radical. Examples are □-caprolactone, β-propiolactone, gamma-butyrolactone and/or methyl-□-caprolactone, 4-hydroxybenzoic acid, 6-hydroxy-2-naphthalenecarboxylic acid or pivalolactone and mixtures thereof. Suitable initiator components are, for example, the low molecular weight dihydric alcohols mentioned above as a component for the polyesterpolyols. The corresponding polymers of alpha-caprolactone are particularly preferred. Lower polyesterdiols or polyetherdiols can also be used as initiators for the preparation of the lactone polymers. Instead of the polymers of lactones, it is also possible to use the corresponding, chemically equivalent polycondensates of the hydroxycarboxylic acids corresponding to the lactones.

Polyester (meth)acrylates can be prepared in a plurality of stages or in one stage, as described, for example, in EP 279 303, from acrylic acid, polycarboxylic acid and polyol.

Re iii)

Suitable saturated thermoplastic polymers are, for example, polymethyl methacrylate, polystyrene, impact-resistant polymethyl methacrylate, high-impact polystyrene, polycarbonate and polyurethanes.

The radiation curability is ensured by the addition of an ethylenically unsaturated, radiation-curable compound. This may be one of the compounds mentioned under i) and/or ii).

The binders (based on the solids content, i.e. without the presence of solvents) have, as a rule, the following composition:
i) at least 20% by weight, preferably at least 30% by weight, particularly preferably at least 50, very particularly preferably at least 60, in particular at least 75 and especially at least 80% by weight and up to 100% by weight, preferably up to 98% by weight, particularly preferably up to 95, very particularly preferably up to 90 and in particular up to 85% by weight,
ii) for example, up to 70% by weight, preferably up to 50% by weight, particularly preferably up to 25% by weight, very particularly preferably up to 10, in particular up to 5% by weight and especially 0% by weight,
ii) for example, up to 50% by weight, preferably up to 25% by weight, particularly preferably up to 10% by weight, very particularly preferably up to 5% by weight and in particular 0% by weight,
with the proviso that the sum is always 100% by weight.

In a further aspect disclosed is a system for producing a formulation using said curable composition associated with a formulation passport or a digital asset

In an embodiment, curable compositions are used for manufacture of a reactive coating or printing ink applied on a solid material which will be cured to form a cured coating or print on such a solid material.

The chemical production network may include one or more one or more chemical and/or mechanical process(es). The chemical production network may produce one or more output material(s) through chemical and/or mechanical processing. The chemical production network may include multiple types of production processes for producing one or more output material(s) from one or more input material(s). The chemical production network may produce one or more output material(s) from input material(s) provided to the chemical production network. The chemical production network may include a complex production network producing multiple chemical products via multiple production process(es). The chemical production network may include connected, interconnected and/or non-connected production process(es). The chemical production network may include a composite or Verbund network.

The chemical production network may include identity preserving or segregated production process(es). Identity preserving or segregated in this context may refer to environmental attribute(s) of input material(s) being preserved or segregated in the production process(es). Examples are non-fossil, e.g. renewable or recycled, input materials used to produce the one or more a curable composition(s) without fossil content. Further examples are fossil input material(s) used to produce the one or more a curable composition(s) with fossil content. Chemical production networks may include non-identity preserving or non-segregated production process(es). Non-identity preserving or non-segregated in this context may refer to non-fossil input material(s) being mixed with fossil input material(s) to produce a curable composition(s). For example, fossil and renewable input materials may be mixed to produce a curable composition(s) with fossil and renewable content.

The chemical production network may include one or more production process(es) with multiple production steps. The production steps included in the chemical network may be defined by the physical system boundary of the chemical production network. The system boundary may be defined by location and/or control over production processes or steps. The system boundary may be defined by a site of the chemical production network. The system boundary may be defined by production process(es) or step(s) controlled by one entity or multiple entities jointly. The system boundary may be defined by the value chain with staggered production process(es) or step(s) to the chemical end product, which may be controlled by multiple entities jointly or separately. The chemical production network may include a waste collection, a sorting step, a recycling step such as pyrolysis, a cracking step such as steam cracking, a separation step to separate intermediates of one process step and further processing steps to convert such intermediates to output material(s), in particular a curable composition(s) leaving the system boundary of the chemical production network. The input material(s) may enter the physical system boundary of the chemical production network. The entry point(s) of the chemical production network may be marked by the entry of input material(s) to the chemical production network or the system boundary of the chemical network. The output material(s), in particular a curable composition(s) may leave the physical system boundary of the chemical production network. The exit point(s) of the chemical production network may be marked by the exit of output material(s), in particular a curable composition(s) from the chemical production network or the system boundary of the chemical network.

The chemical production network may include one or more production chain(s) for the production of a curable composition. The production chain(s) for the production of a curable composition may be interconnected. The production chain(s) for the production of a curable composition may be interconnected with production chain(s) for the production of other output material(s). The production chain(s) for the production of a curable composition may include production chain(s) for the production of intermediates used to produce a curable composition. The production chain(s) for the production of a curable composition may use input material(s) provided by chemical network(s) for the production of intermediates usable to produce a curable composition.

One or more input material(s) may be provided to the chemical production network for producing one or more output material(s), in particular a curable composition(s). The output material(s), in particular a curable composition(s) may be produced from one or more input material(s) through one or more chemical process(s) of the chemical production network. The input material may comprise any input material entering the chemical production network at any entry point. The input material may include an output material(s), in particular a curable composition(s) such as a natural, organic or inorganic output material(s), in particular a curable composition(s). The input material may be a pre-cursor product, an intermediate material, a feedstock or a raw material used to produce one or more output material(s), in particular a curable composition(s). The input material may be fed to the chemical production network to produce one or more output material(s), in particular a curable composition(s). The input material may be fed to chemical production network including one or more production process(es) with multiple process steps. The input material may be fed to the chemical production network at the start of the production process or at any intermediate stage of the production process. The input materials entering the chemical production network may be used to produce one or more output material(s), in particular a curable composition(s).

The input material may be associated with an input material identifier. The input material identifier may comprise any identifier uniquely associated with the input material. The input material identifier may be associated with the physical entity of the input material. The input material identifier may be associated with a single batch of input material. The input material identifier may be associated with a group of input materials. The identifier may be associated with multiple physical entities of the input material. The input material identifier may be associated with continuous or semi-continuous stream of input material. The input material identifier may be associated with a stream of the input material e.g. over a certain time period or from a certain supplier. The input material identifier may be linked or connected to one or more environmental attribute(s).

Environmental attribute may refer to a property related to the environmental impact. Such property may be the property of input material(s), chemical process(es), chemical production network(s) and/or a curable composition(s). The environmental attribute may indicate an environmental performance of input material(s), chemical process(es), chemical production network(s) and/or a curable composition(s). The environmental attribute may be derived from properties of input material(s), chemical process(es), chemical production network(s) and/or a curable composition(s). The environmental attribute may be associated with the environmental impact of input material(s), chemical process(es), chemical production network(s) and/or a curable composition(s) at any stage of the lifecycle of a curable composition(s). The stages may include providing raw material, providing feedstock, producing chemical products, such as intermediate products or end products, producing discrete products by using the chemical products, using chemical products or discrete products, treating end-of-life products, recycling end-of-life products, disposing end-of-life products, reusing components from end-of-life products or any subset of stages. The environmental attribute may be specified or may be derived from any activity of one or more entities participating at any stage of the lifecycle of one or more material(s) or product(s).

The environmental attribute may include one or more characteristic(s) that are attributable to environmental impact of input material(s), chemical process(es), chemical production network(s) and/or a curable composition(s). The environmental attribute may include environmental, technical, recyclability or circularity characteristics(s) associated with the environmental impact of input material(s), chemical process(es), chemical production network(s) and/or a curable composition(s).

The environmental attribute may include one or more characteristic(s) that are attributable to the environmental impact of input material(s), chemical process(es), chemical production network(s) and/or a curable composition(s). The environmental attribute may include environmental, technical, recyclability, safety, legal, or circularity characteristics(s), preferably environmental, technical, recyclability, circularity characteristics(s) associated with the environmental impact of input material(s), chemical process(es), chemical production network(s) and/or a curable composition(s). The one or more environmental attribute(s) may be attributable to the environmental impact of a curable composition. The one or more environmental attribute(s) may relate to environmental, technical, recyclability, circularity and/or complementary risk characteristic(s) of a curable composition.

Environmental characteristic(s) may specify or quantify ecological or compositional criteria associated with environmental impact. Environmental characteristic(s) may be or may be derived from measurements taken during the lifecycle. Environmental characteristics may be determined at any stage of the lifecycle and may characterize the environmental impact for such stage or up to such stage. Environmental characteristic(s) may for example include carbon footprint, greenhouse gas emissions, resource usage, air emissions, ozone depletion potential, water pollution, noise pollution, energy consumption, waste reduction, or eutrophication potential. Environmental characteristic(s) may for example include product characteristics like compositions, impurity profiles, or information on substances of concern as defined by industry specific regulatory or value chain requirements or product characteristics related to the production of the product like bio based, vegetable based, animal based, halogen-free, fluorine-free, vegan, halal, kosher, palm oil-free, natural, free of hazardous substances, volatile organic compounds-free or any combinations thereof. Preferably environmental characteristic(s) include product characteristics related to the production of the product like bio based, vegetable based, animal based, halogen-free, fluorine-free, vegan, halal, kosher, palm oil-free, natural, free of hazardous substances, volatile organic compounds-free or any combinations thereof.

Technical characteristic(s) may specify or quantify performance at least indirectly associated with the environmental impact. Technical characteristic(s) may be or may be derived from measurements taken during the lifecycle. Technical characteristics may be determined at any stage of the lifecycle and may characterize the performance for such stage or up to such stage. Technical characteristic(s) may for example include chemical composition data, raw material composition such as bio-based or recycled input material content specifying e.g. x % non fossil and y % fossil content, bill of materials, product specification data such as product purity, product form (as indication to their impact on dust formation/release), safety data, product extractability, migration data, toxicological data or ecotoxicological data, product component data, data needed to assess safe applications or application instructions, quality data or any combinations thereof.

Circularity characteristic(s) may specify or quantify the life cycle characteristics associated with circular uses. Circularity characteristic(s) may be or may be derived from measurements taken during the lifecycle. Circularity characteristic(s) may be or may be derived from circular data recorded in one or more prior lifecycle(s) including reuse. Circularity characteristic(s) may be determined at any stage of the lifecycle and may characterize the reuse or recycling performance for such stage or up to such stage. Circularity characteristic(s) may for example include recycling data, reuse rate, recycling rate, recycling loops, reuse performance, reused quality or any combinations thereof.

Recyclability characteristic(s) may specify or quantify life cycle characteristics associated with recycling uses. Recyclability characteristic(s) may include the composition of the material including specifically tailored constituents making the material suitable for recycling. Recyclability characteristic(s) may be or may be derived from measurements taken during the lifecycle. Recyclability characteristic(s) may be or may be derived from recycling data recorded in one or more prior lifecycle(s). Recyclability characteristics may be determined at any stage of the lifecycle and may characterize the recycling performance for such stage or up to such stage. Recyclability characteristic(s) may for example include recycling data, number of reuses, recyclate composition, recyclate quality, waste stream composition, waste stream quality or any combinations thereof.

Environmental impact may for example include impacts on the safe use and application and compliance with mandatory and/or voluntary assessment schemes needed for the application or required by the value chain in which the product itself, formulation of said product and conversion products of said products are handled.

In one embodiment a curable composition passport or digital asset of a curable composition may include mass balanced environmental attributes related to the input material. Mass balanced environmental attributes may include environmental attributes of the input material(s) used to produce a curable composition, which are tracked and by mass attributable to a curable composition. The environmental impact of input material(s) may be determined based on input material(s) used in the chemical process(s) to produce a curable composition. For example, bio-based, renewable and/or recycled content of input material(s) used to produce a curable composition may be tracked. Further for example, castor oil or palm oil content of input material(s) used to produce a curable composition may be tracked. Further for example, properties related to the environmental impact of the input material include RSPO palm oil, palm oil free or castor oil may be tracked. Further for example, properties of the chemical process(es) used to produce a curable composition may be tracked. Examples of tracked process properties related to the environmental impact include water consumption, CO₂ emissions and/or Greenhouse Gas (GHG) emissions, amount of waste generation, mixed material generation, design for recycling, energy consumption, processing properties such as less waste or less loss of properties. The properties may be tracked based on a certificate from a certifying agency. The properties may be tracked based on inherent physical properties derived from measurements.

In one embodiment the produced a curable composition is connected to the decentral identifier physically identifying the produced a curable composition. The production operating apparatus may be configured to provide the decentral identifier associated with a physical entity of the produced a curable composition. The production operating apparatus may be configured to link the decentral identifier to a physical identifier of the produced a curable composition. The production operating apparatus may be configured to assign the decentral identifier to the physical identifier connected to the produced a curable composition.

In one embodiment the decentral identifier relates to data associated with at least one product produced from a curable composition, wherein the one or more environmental attribute(s) associated with the at least one product is derived from one or more environmental attribute(s) associated with a curable composition. The one or more environmental attribute(s) associated with a curable composition may be associated with the one or more input material(s) and/or the chemical process(s) used to produce a curable composition. The decentral identifier may relate to any identifier uniquely associated with a curable composition. The decentral identifier may be associated with the physical entity of a curable composition. The decentral identifier may refer to a single batch of a curable composition. The decentral identifier may be associated with a group of a curable composition. The identifier may refer to multiple physical entities of a curable composition. The decentral identifier may be associated with continuous or semi-continuous stream of a curable composition. The identifier may refer to a stream of a curable composition e.g. over a certain time period or from a certain supplier.

In one embodiment the one or more environmental attribute(s) associated with a curable composition(s) are provided from at least one balancing account configured to store environmental attribute(s) associated with input material(s). An inbound allocator may be configured to allocate the one or more environmental attribute(s) associated with input material(s) to at least one balancing account e.g. on entry of the input material to the chemical production network. The balancing account may be associated with the respective environmental attribute type. An outbound assigner may be configured to assign at least one environmental attribute from the at least one balancing account associated with the respective environmental attribute to the decentral identifier. One or more environmental attribute(s) may be assigned to the at least one decentral identifier. Assignment may include de-allocation of the one or more environmental attributes from the balancing account associated with the respective environmental attribute type. By using the balancing accounts environmental attributes of input materials can be reliably tracked and assigned to a curable composition.

In one embodiment the one or more environmental attribute(s) associated with a curable composition(s) are provided from at least one balancing account configured to store environmental attribute(s) associated with input material(s). An inbound allocator may be configured to allocate the one or more environmental attribute(s) to at least one balancing account associated with the respective environmental attribute, e.g. on input material entering the chemical production network. The balancing account may be associated with the respective environmental attribute type. The one or more environmental attribute(s) associated with the input materials may be allocated to the balancing account associated with the respective environmental attribute type. An outbound assigner may be configured to assign or link at least one environmental attribute from the at least one balancing account associated with the respective environmental attribute type to the decentral identifier. This may include de-allocation of the one or more environmental attribute(es) from the balancing account associated with the respective environmental attribute type. By using the balancing accounts environmental attributes can be tacked through the chemical production network. This way the environmental attributes may be detached from the material flow. Attribution of environmental attribute(s) may be conducted on a mass balance basis for the chemical production network. In such approach the total mass of input materials and a curable composition as well as the attribution of respective environmental attribute(s) associated with input materials and a curable composition are balanced.

In one embodiment the one or more environmental attribute(s) are associated with at least one property related to the environmental impact of the one or more input material(s) and/or the chemical process(s). The one or more environmental attribute(s) may specify environmental properties of the input material(s) used to produce a curable composition and/or the one or more environmental attribute(s) may specify environmental properties of the chemical process(es) used to produce a curable composition. The one or more environmental attribute(s) may be generated from environmental properties of the input material(s) used to produce a curable composition, process data associated with the chemical processing of the input material(s) and/or energy data associated with the energy consumption of the chemical processing. The one or more environmental attribute(s) may include a recycled content associated with the input material(s) and allocated or allocatable to a curable composition(s), a renewable content associated with the input material(s) and allocated or allocatable to a curable composition(s), and/or a product carbon footprint associated with a curable composition(s).

In one embodiment the production operating apparatus is configured to gather environmental attributes associated with the produced a curable composition before, during and/or after production of a curable composition by the chemical production network. The environmental attributes associated with the produced a curable composition may relate to input material(s). The environmental attributes associated with input materials may be provided before, during and/or after production of a curable composition by the chemical production network. The environmental attributes associated with input materials may be allocated to at least one balancing account before, during and/or after production of a curable composition by the chemical production network. The environmental attributes associated with the produced a curable composition may relate to environmental properties generated from process data associated with the chemical processing of the input material(s) and/or energy data associated with the energy consumption of the chemical processing. The environmental attributes associated with the produced a curable composition may be generated before, during and/or after production of a curable composition by the chemical production network. The process data associated with the chemical processing of the input material(s) and/or energy data associated with the energy consumption of the chemical processing may be gathered prior, during and/or after production of a curable composition.

In one embodiment a curable composition passport or the digital asset include the decentral identifier associated with a curable composition and the one or more environmental attribute(s) linked to the decentral identifier. The one or more environmental attribute(s) may be linked to the decentral identifier included a curable composition passport or the digital asset. The one or more environmental attribute(s) may be stored in a data base of a curable composition producer for access by any a curable composition user. The one or more environmental attribute(s) may be stored in a data base of a curable composition producer for transfer to a curable composition user e.g. when accessed or on providing a curable composition. The decentral identifier may comprise any unique identifier uniquely associated with a curable composition producer and a curable composition data such as the environmental attributes. The decentral identifier may include a Universally Unique IDentifier (UUID) or a Digital IDentifier (DID). The decentral identifier may be issued by a central or decentral identity issuer. The decentral identifier may be linked to authentication and/or authorization information. Via the decentral identifier and its unique association with a curable composition producer and a curable composition data, such as the environmental attributes, access to a curable composition data may be controlled by a curable composition producer. This contrasts with central authority schemes, where identifiers are provided by such central authority and access to data is controlled by such central authority. Decentral in this context refers to the usage of the identifier in implementation as controlled by the data owner, such as a curable composition producer.

The decentral identifier may be uniquely associated with a curable composition or the physical entity of a curable composition, e.g. as packaged for transportation to a consumer of the curable composition. The decentral identifier may be uniquely to the one or more environmental attribute(s). A curable composition passport or the digital asset may include one or more digital representation(s) pointing to a curable composition data including the environmental attribute(s) or parts thereof including the environmental attribute(s). The digital representation may comprise at least one interface to a data providing service. It may further include at least one interface to a data consuming service. It may include an endpoint for data exchange or sharing (resource endpoint) or an endpoint for service interaction (service endpoint), that is uniquely identified via a communication protocol. The digital representation(s) pointing to a curable composition data or parts thereof may be uniquely associated with the decentral identifier.

A curable composition passport or the digital asset may comprise or be connected to a digital representation of a curable composition data, such as environmental attribute(s). The digital representation may include a representation for accessing a curable composition data, such as environmental attribute(s) or part thereof. The digital representation may include a representation of a curable composition data, such as environmental attribute(s). A curable composition passport or the digital asset may include or be connected to data related to a curable composition data, such as environmental attribute(s), the authentication information and the decentral identifier. The data related to a curable composition data, such as environmental attribute(s) may include the digital representation of a curable composition data, such as environmental attribute(s).

### BRIEF DESCRIPTION OF THE DRAWINGS

In the following, the present disclosure is further described with reference to the enclosed figures:

FIG. 1 illustrates schematically an example of a chemical production network producing one or more output material(s) from one or more input material(s) in connection with a production operating system including an attribute management system.

FIG. 2 illustrates schematically an example of attributing environmental attributes of input materials to output materials of the chemical production network.

FIG. 3 illustrates schematically an example of attributing environmental attributes of input materials and chemical processes to an output material of the chemical production network.

FIG. 4 illustrates schematically another example of a method or apparatus for providing environmental attributes associated with output materials to a material user as data consumer via a decentral network.

FIG. 5 illustrates schematically an example of a method or apparatus for providing environmental attributes of output materials across value chains via the decentral network.

FIG. 6 illustrates schematically an example of a chemical production network for producing a curable composition associated with the digital asset.

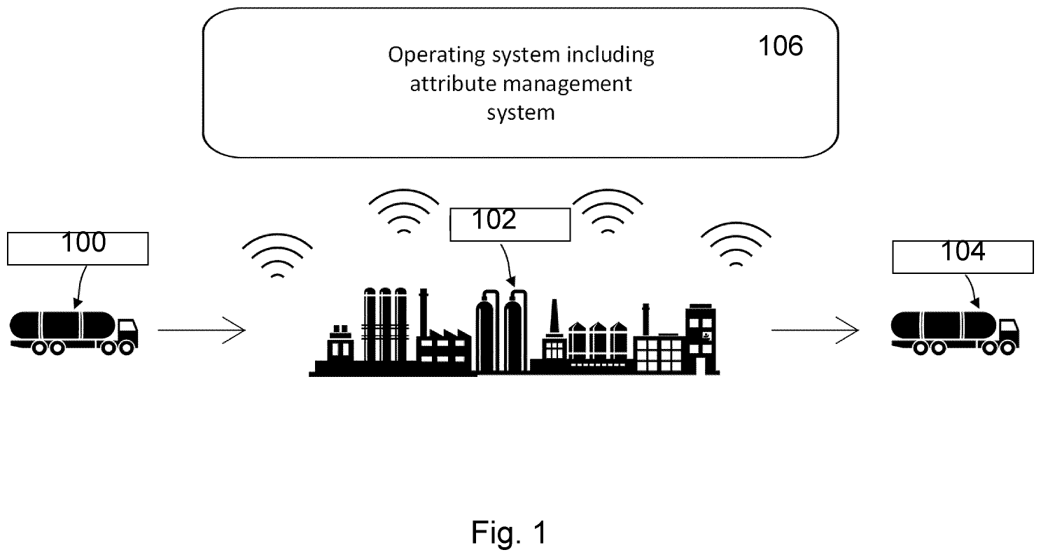

FIG. 1 illustrates an example of a chemical production network 102 producing one or more output material, in particular a curable composition(s) 104 from one or more input material(s) 100 in connection with a production operating system 106 including an attribute management system.

For producing one or more output material(s), in particular a curable composition(s) 104 different input materials 100 may be provided as physical inputs to the chemical production network 102. The physical input and output material(s), in particular a curable composition(s) 100, 104 may be associated with one or more properties related to environmental impact. The properties related to environmental impact may be digitalized in the form of environmental attributes such as recycled or bio-based content of the input materials. The production operating system 106 may be configured to ingest such environmental attributes and to track the environmental attributes across the chemical production network 102 from input materials 100 to output material(s), in particular a curable composition(s) 104.

The chemical production network 102 may include multiple interlinked processing steps. The chemical production network 102 may be an integrated chemical production network 102 with interrelated production chains. The chemical production network 102 may include multiple different production chains that have at least one intermediate product in common. The chemical production network 102 may include multiple stages of the chemical value chain. The chemical production network 102 may include the producing, refining, processing and/or purification of gas or crude oil. The chemical production network 102 may include a stream cracker, or a syngas plant connected to multiple production chains that output chemical products 104 from the effluent of such plants. The chemical production network 102 may include multiple production chains that output from one or more input material(s) 100 one or more output material(s), in particular a curable composition(s). The chemical production network 102 may include multiple tiers of a chemical value chain. The chemical production network 102 may include a physically interconnected arrangement of production sites. The production sites may be at the same location or at different locations. In the latter case the production sites may be interconnected by means of dedicated transportation systems such as pipelines, supply chain vehicles, like trucks, supply chain ships or other cargo transportation means.

The chemical production network 102 may chemically convert input materials 100 to one or more output material(s), in particular a curable composition(s) 104. The chemical production network 102 may convert input materials 100 by way of chemical conversion to one or more output material(s), in particular a curable composition(s) 104.

The input materials 100 may be fed to the chemical production network 102 at any entry point. The input materials 100 may be fed to the chemical production network 102 at the start of the chemical production network 102. Input materials 100 may for example make up the feedstock of a steam cracker. The input material 100 may include non-fossil input material, such as bio-based or recycled material, and/or fossil input material for the manufacture of chemical intermediates and chemical output material(s), in particular a curable composition(s) 104.

The chemical production network 102 may include multiple production steps. The production steps included in the chemical production network 102 may be defined by the system boundary of the chemical production network 102. The system boundary may be defined by location or control over production processes. The system boundary may be defined by the site of the chemical production network 102. The system boundary may be defined by production processes controlled by one entity or multiple entities jointly. The system boundary may be defined by value chain with staggered production processes to an end product, which may be controlled by multiple entities separately. The chemical production network 102 may include a waste collection and sorting step, a recycling step such as pyrolysis, a cracking step such as steam cracking, a separation step to separate intermediates of one process step and further processing steps to convert such intermediates to output material(s), in particular a curable composition(s) 104 leaving the system boundary of the chemical production network 102.

The production operating system 106 of the chemical production network 102 may be configured to monitor and/or control the chemical production network 102 based on operating parameters of the different processes. One process step monitored and/or controlled may be the feed of input materials 100 or the release of output material(s), in particular a curable composition(s) 104. Another process step monitored and/or controlled may be the registration of environmental attributes associated with input materials 100 entering the system boundary of the chemical production network 102. Yet another process step monitored and/or controlled may be the attribution of environmental attributes to output material(s), in particular a curable composition(s) 104 produced via the chemical production network. Yet another process step monitored and/or controlled may be the management of environmental attributes associated with input materials 100 and output material(s), in particular a curable composition(s) 104 of the chemical production network 102.

The production operating system 106 may be configured to register inbound environmental attributes, to assign outbound environmental attributes. The production operating system 106 may be configured to access data related the inputs materials 100, the processes and/or the output material(s), in particular a curable composition(s) 104 of the chemical production network 102. For example, the production operating system 106 may be configured to register a recycled or bio-based content of the one or more input material(s) 100 used in the chemical production network 102 as environmental attribute. The production operating system 106 may be configured to allocate the environmental attribute to at least one balancing account associated with the recycled or bio-based content of the input materials 100. The production operating system 106 may be configured to allocate at least a part of the environmental attributes from the at least one balancing account to the at least one output material(s), in particular a curable composition(s) 104.

The production operating system 102 may be configured to handle environmental attributes related to the input and output material(s), in particular a curable composition(s) 100/104 of the chemical production network 102. For example, the production operating system 106 may be configured to determine environmental attributes associated with the use of input materials 100 impacting the environmental property of the chemical production network 102 and the output material(s), in particular a curable composition(s) 104 produced by the chemical production network 102. Further in particular, the production operating system 102 may be configured to determine environmental attributes associated with the output material(s), in particular a curable composition(s) 104. This way the production operating system 102 may be configured to store environmental attributes in balancing accounts or to delete environmental attributes from the balancing accounts. The environmental attributes may hence be viewed as a credit that may be deposited in an account or deducted from an account related to the input and output material(s), in particular a curable composition(s) of the chemical production network 102. This way the environmental impact of the production may be tracked and/or traced.

In chemical production networks 102 multiple value chains may be linked. Additionally different input materials 100 or chemical processes impacting the environmental property of output material(s), in particular a curable composition(s) 104 produced by the chemical production network 102 may be used. Examples of input materials 100 impacting at least one environmental property of output material(s), in particular a curable composition(s) 104 produced from such input materials 100 are recycled, renewable or bio-based input materials 104. Examples of chemical processes impacting the environmental property include chemical processes using environmentally friendly technology such as carbon capture, carbon utilization or heat pumps.

Owing to the processing of chemicals in continuous or semi-continuous production and the complexity of chemical production networks 102, traceability of the input materials through the network may be hampered. In such scenarios, an equivalent environmental attribute signifying the impact on the environmental property of output material(s), in particular a curable composition(s) 104 produced by the chemical production network may be allocated to balancing accounts 122 and assigned to one or more output material(s), in particular a curable composition(s) 104 of the chemical production network 102. The environmental attributes may hence be decoupled from the physical material flow inside the chemical production network 102. Decoupling may be based on the mass balance model in that the equivalent amount assigned to the one or more output material(s), in particular a curable composition(s) may not exceed the equivalent amount provided by input materials or processes. If an equivalent amount has been allocated to the virtual account of one environmental attribute type, it may not be allocated a second time to another virtual account of the one environmental attribute type. Environmental attribute types may be recycled, bio-based, renewable or the like. Environmental attributes may be provided in the form of digital assets or curable composition passports attached to the physical entity of a curable composition.

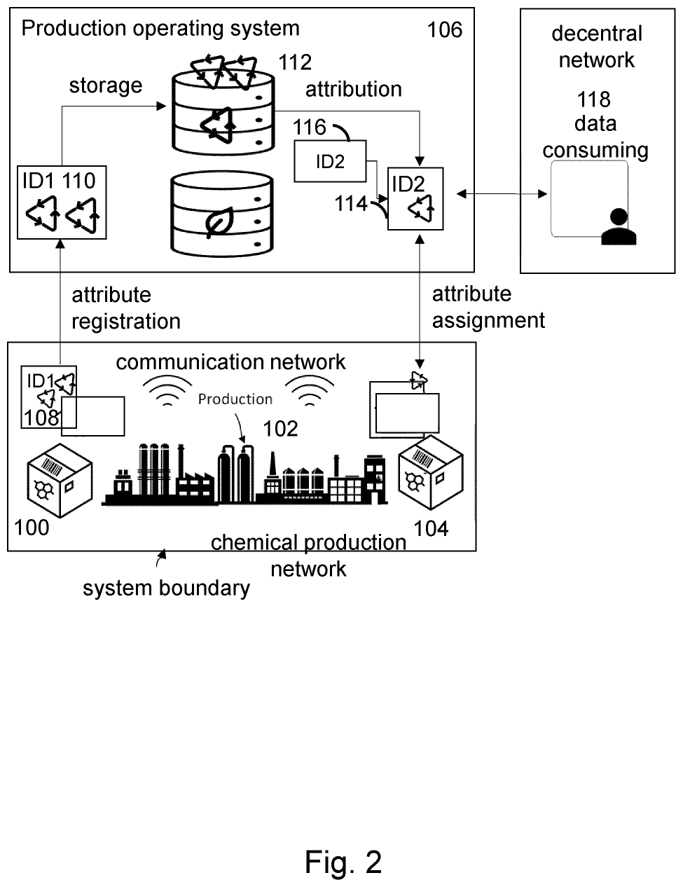

FIG. 2 illustrates schematically an example of attributing environmental attributes associated with input materials 100 to output material(s), in particular a curable composition(s) 104 of the chemical production network 102.

As shown in FIG. 1 the chemical production network 102 and operations of the chemical production network 102 may be monitored and/or controlled by a production operating system 106. The production operating system 106 may be configured to track environmental attributes from input materials 100 fed to the chemical production network 102 to output material(s), in particular a curable composition(s) 104 produced by the chemical production network 102. For tracking the operating system 106 may be configured to register environmental attributes associated with the input materials 100 provided to the chemical production network 102 and to attribute environmental attributes to output material(s), in particular a curable composition(s) 104 produced by the chemical production network 102.

The input materials 100 such as pyrolysis oil, bio-naphtha or bio-gas may be provided to the chemical production network 102. The input materials 100 may enter the chemical production network at the entry point, such as a such as a steam cracker or a syngas plant. The input materials 100 may be used in the chemical production network 102 to produce one or more output material(s), in particular a curable composition(s) 104 from the input materials 100. output material(s), in particular a curable composition(s) 104 may be provided at exit points of the chemical production network 102. Further output material(s) may be (Meth)acrylates, Isocyanates, Epoxides.

On entry of the input material 100, input material data 108 may be provided via a communication network to a computing interface of the production operating system 106. A data provider, such as a QR code reader, may be configured to provide material data 108 related to the one or more input material(s) 100 and respective environmental attributes 108 to a computing interface configured to allocate the environmental attributes associated with the input materials 100. The material data 108 may include the input material identifier and environmental attributes associated with the input materials 100. The input material identifier may be associated with the physical entity of the input material 100 entering the chemical production network 102. The material data may be provided on, prior or after providing of the one or more input material(s) at entry points to the chemical production network 102.

The input material identifier may be linked to the environmental attribute(s) associated with the respective input material(s) 100, the amount of input material 100 and the certificate certifying the environmental attribute(s). The amount of input material may be a measured amount of input material 100 fed to a plant or storage of the chemical production network 102 for producing one or more output material(s), in particular a curable composition(s) 104 from the input material(s) 100. The input material identifier associated with the respective input material 100, the environmental attribute(s) associated with the respective input material(s) 100 and the amount of input material(s) 100 provided to the chemical production network 102 may be provided to the production operating system 106. Such data may be provided via a communication network on entry to chemical production network 102, or the data may be transferred from a computing system to the production operating system 106.

An inbound allocator 110 may be configured to allocate the one or more environmental attribute(s) to at least one balancing account 112 associated with the respective environmental attribute. For example, one balancing account 112 may relate to environmental attributes from recycled material and another balancing account 112 may relate to environmental attributes from bio-based material. The balancing account may be associated with the respective environmental attribute type, such as bio-based or recycled 100. Based on such association the balancing account associated with the environmental attribute type of the respective input material 100 may be selected. The environmental attributes may be allocated to the selected balancing account. For example, the account 112 for recycled material may be selected and the environmental attribute may be allocated to such account 122.

To allocate, the one or more environmental attribute(s) may be converted to balancing units and the balancing units may be allocated to the balancing account 122. The conversion may be based on a conversion factor such as mass, weight, carbon atoms, hydrogen atoms, methane equivalents or any other suitable measure for quantifying the environmental impact of the environmental attribute. The conversion factor may hence take into account the difference between producing chemical products from conventional input material(s) and producing chemical products from non-conventional input material(s) or producing chemical products from a mix of conventional and non-conventional input materials. The conversion factor may relate to differences in chemical and/or physical properties of conventional and non-conventional input material(s).

By using the balancing accounts 112 it can be ensured that environmental attributes of input materials 100 are only used once for assignment to a curable composition 104. This way double counting on input or output is avoided to ensure positive environmental impact can be reliable tracked and assigned to a curable composition 104.

An identifier provider 116 may be configured to provide a curable composition identifier associated with a curable composition produced by the chemical production network 102 and provided at the exit point from the chemical production network 102.

An outbound assigner 114 may be configured to assign at least one environmental attribute from the at least one balancing account 112 associated with the respective environmental attribute to a curable composition identifier ID2. One or more environmental attribute(s) may be assigned to the at least one a curable composition identifier ID2. Assignment may include de-allocation of the one or more environmental attributes from the balancing account 112 associated with the respective environmental attribute type. Assignment may include converting one or more balancing unit(s) to one or more environmental attribute(s).

Assigning at least one environmental attribute associated with input material(s) to a curable composition(s) may include the linking of a curable composition identifier ID2 with the environmental attribute. A curable composition identifier ID2 may be associated with the physical entity of a curable composition. This way the virtual identifier of a material may be uniquely linked to the physical material. Such linking may include a physical or virtual link of identifiers uniquely associated with the physical material. For physical linking a tag or code may be physically connected to the material, e.g., by printing a QR code on the packaging. For virtual linking different identifiers associated with the physical material may be linked. For example, an order number, a batch number, LOT number or a combination thereof may be linked.

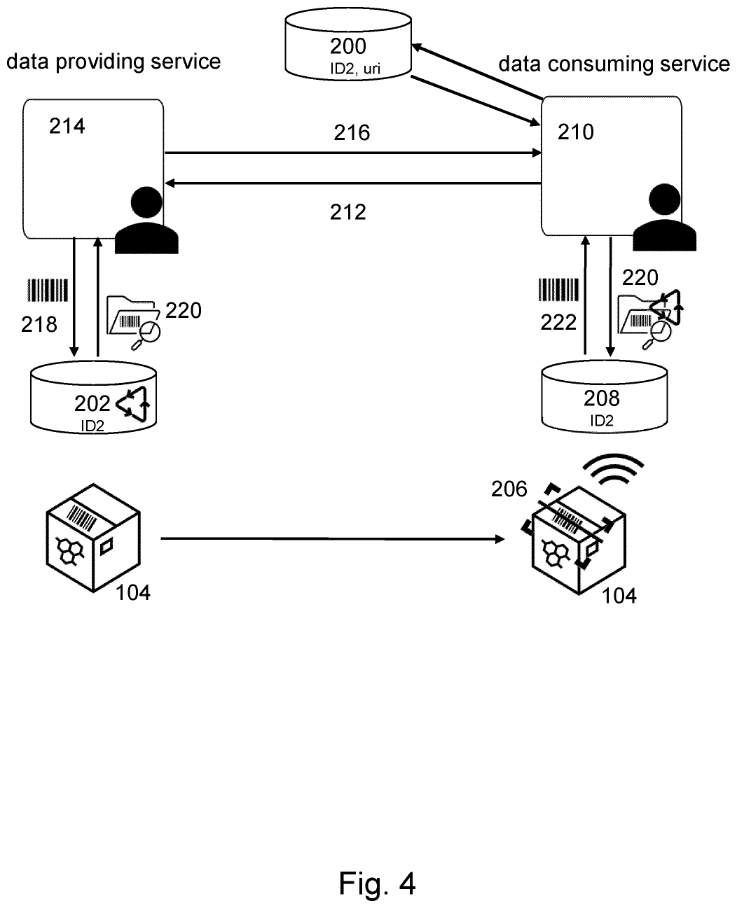

The outbound assigner 114 may be configured to provide the environmental attributes associated with a curable composition to a data consumer, such as a system associated with a user of a curable composition. The outbound assigner 114 may be configured to provide the environmental attributes associated with a curable composition to a decentral network as will be described in the example of FIG. 4. Environmental attributes may be provided via the above ID based schema in the form of digital assets or curable composition passports associated with the physical entity of a curable composition.

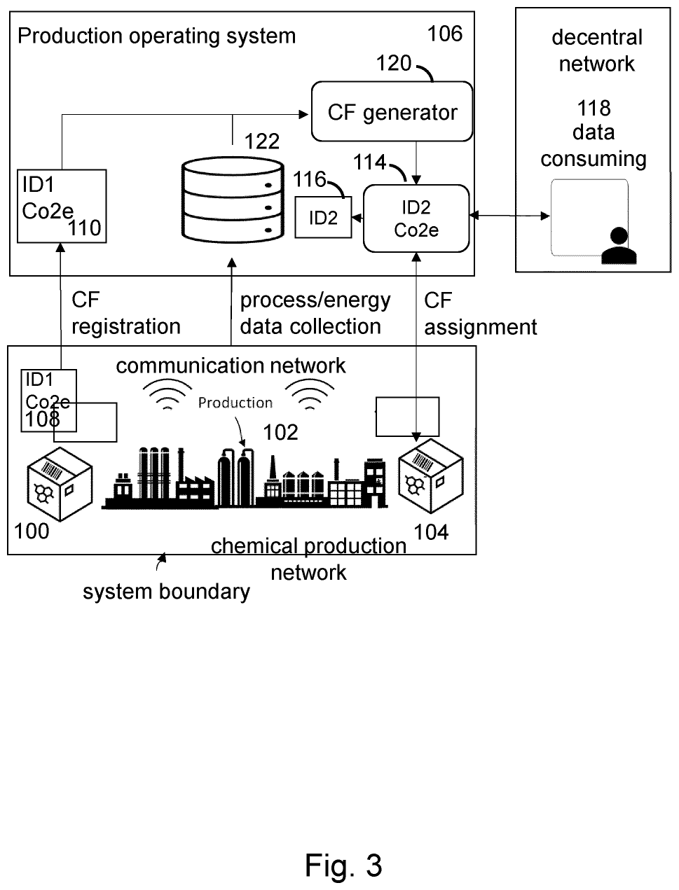

FIG. 3 illustrates schematically an example of attributing environmental attributes of input materials 100 and chemical processes to a curable composition 104 of the chemical production network 102.

As described in the context of FIGS. 1 and 2 the chemical production network 102 and operations of the chemical production network 102 may be monitored and/or controlled by a production operating system 106. Input materials 100 such as pyrolysis oil, bio-naphtha or bio-gas may be provided to the chemical production network 102. The input materials 100 may be used in the chemical production network 102 to produce one or more a curable composition(s) 104 from the input materials 100.

On entry of the input material 100, input material data 108 may be provided via a communication network to a computing interface of the production operating system 106. A data provider, such as a QR code reader, may be configured to provide material data 108 related to the one or more input material(s) 100 and respective environmental attributes 108 to a computing interface configured to allocate the environmental attributes associated with the input materials 100. The material data 108 may include the input material identifier and environmental attributes associated with the input materials 100. The input material identifier may be associated with the physical entity of the input material 100 entering the chemical production network 102. The input material identifier may be linked to the carbon footprint of the input material 100 as environmental attribute. The material data may be provided on, prior or after providing of the one or more input material(s) at entry points to the chemical production network 102.

The inbound allocator 110 may be configured to retrieve the one or more environmental attribute(s) and to provide such attributes to the carbon footprint (CF) generator 120. A process data provider 122 may be configured to gather process data associated with the chemical processing of the input material(s) 100 to produce a curable composition(s) 104. The process data provider 122 may be configured to gather energy data associated with the energy consumption of the chemical processing. The process data provider 122 may be configured to provide the process data and the energy data to the CF generator 120.

The CF generator 120 may be configured to determine the carbon footprint of a curable composition produced by the chemical production network. The carbon footprint of the of a curable composition may be determined based on the process data, the energy data and the carbon footprint of the input material(s) 100 used to produce a curable composition.

An identifier provider 116 may be configured to provide a curable composition identifier associated with a curable composition produced by the chemical production network 102 and provided at the exit point from the chemical production network 102.

An outbound assigner 114 may be configured to assign the determined carbon footprint to a curable composition identifier ID2. One or more environmental attribute(s) may be assigned to the at least one a curable composition identifier ID2, such as described in the context of FIG. 2.

The outbound assigner 114 may be configured to provide the environmental attributes, in particular the carbon footprint, associated with a curable composition to a data consumer, such as a system associated with a user of a curable composition. The outbound assigner 114 may be configured to provide the environmental attributes associated with a curable composition to a decentral network as will be described in the example of FIG. 4. Environmental attributes may be provided via the above ID based schema in the form of digital assets or curable composition passports associated with the physical entity of a curable composition.

FIG. 4 illustrates schematically an example of a method or apparatus for providing environmental attributes associated with a curable composition to a material user as data consumer via a decentral network.

A curable composition 104 as produced by the chemical production network 102 may be provided in association with the digital asset as described in the context of FIGS. 2 and 3. The digital asset may include a curable composition identifier. The digital asset may include one or more environmental attribute(s) such as the product carbon footprint, recycled content or bio-based content. The digital asset may relate to one or more environmental attribute(s) such as the product carbon footprint, recycled content or bio-based content. The digital asset may include a digital representation of one or more environmental attribute(s) such as the product carbon footprint, recycled content or bio-based content.

The digital asset may further include or relate to authentication and/or authorization information linked to a curable composition identifier. The authentication and/or authorization information may be provided for authentication and/or authorization of a data provider 208 and/or data consumer 210. A curable composition identifier may include or relate to a decentral identifier, that is uniquely associated with a curable composition. The decentral identifier may be connected to the digital representation of the environmental attributes. The digital representation may include a representation for accessing the environmental attributes or parts thereof. The decentral identifier may include a Universally Unique IDentifier (UUID) or a Digital IDentifier (DID). The decentral identifier may include any unique identifier uniquely associated with a data owner and/or a curable composition. The data owner may be the producer of a curable composition. Via the decentral identifier and its unique association with the data owner and/or a curable composition access to the material configuration data may be controlled by the data owner.

The digital asset including the digital representation of one or more environmental attribute(s) such as the product carbon footprint, recycled content or bio-based content may be stored in a decentral data base 200. The one or more environmental attribute(s) such as the product carbon footprint, recycled content or bio-based content may be stored in a data base 202 associated with the data owner, such as the producer of a curable composition 104.

A curable composition 104 may be physically delivered to a user of a curable composition. A curable composition may be connected with a QR-code having encoded a curable composition identifier. The user of a curable composition may read the QR-code through a QR-code reader 206. A curable composition identifier may be provided to a data base 208 associated with the user of a curable composition 104. In other embodiments the user of a curable composition may retrieve a curable composition identifier through the decentral data base 200.

The data owner in this example may be the input material producer, the output material producer, the output material user, the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service may comprise computer-executable instructions for accessing and/or processing data, such as a curable composition data associated with the data owner.

The data providing service may comprise computer-executable instructions for providing and/or processing data, such as a curable composition data associated with the data owner for accessing and/or processing by the data consuming service.

Based on the received a curable composition identifier a request to access the environmental attributes associated with a curable composition identifier may be triggered by the data consuming service 210 as signified by arrow 212. A curable composition identifier may be provided to the data providing service 214 of the producer of a curable composition 104. In addition, authentication and/or authorization information may be provided.

The request may be authenticated and/or authorized to access the environmental attributes associated with a curable composition identifier. Based on successful authorization and/or authentication access to the environmental attributes associated with a curable composition identifier may be granted.

For access a curable composition identifier may be provided to the data providing service 214 as signified by arrow 212. The data providing service 214 may use the received a curable composition identifier to retrieve the environmental attributes associated with a curable composition 104 as signified by arrows 218 and 220. The environmental attributes associated with a curable composition 104 provided to the data providing service 214 may be provided to the data consuming service 210 as signified by arrow 216. The environmental attributes associated with a curable composition 104 may be stored in the data base 208 associated with the user of a curable composition 104 as signified by arrow 220.

Through the output identifier or decentral identifier, the environmental attributes can be uniquely associated with a curable composition. Through the decentral network the environmental attributes may be transferred between the producer of a curable composition and the user of a curable composition. This way the environmental attributes can be shared with unique association to a curable composition and without central intermediary directly between the value chain players. This allows for transparency of environmental attributes across the value chain and positive environmental impacts from a curable composition of the chemical production network can be tracked through the value chain.

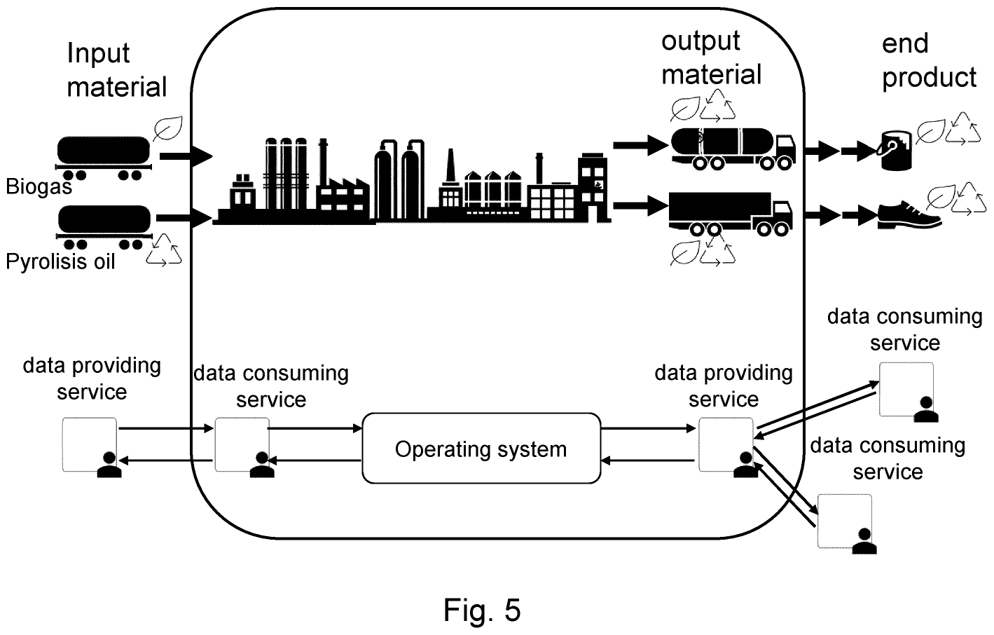

FIG. 5 illustrates schematically an example of a method or apparatus for providing environmental attributes associated with a curable composition across value chains via the decentral network.

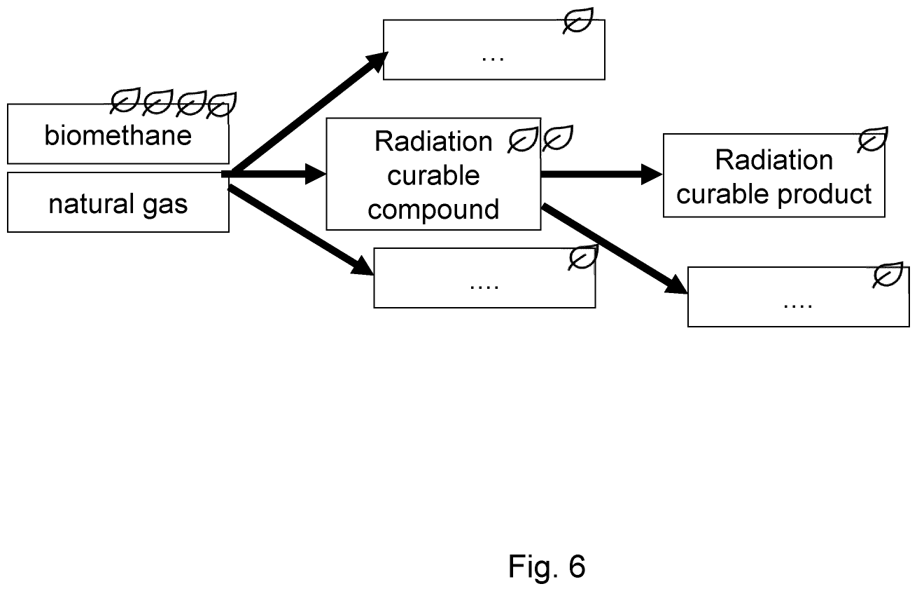

In the example of FIG. 6 a fully connected value chain including the chemical production network is illustrated. In the example, the input material provider, a curable composition producer, a curable composition user and the end product producer may be connected to the decentral network as described in the context of FIG. 4. Environmental attributes may be provided via the ID based schema described in the context of FIGS. 2-5 in the form of digital assets or curable composition passports associated with the physical entity of the input material, a curable composition, any intermediate product or the end product.

The input material provider may provide the input materials such as bio-gas or pyrolysis oil. The environmental attributes of the input material may be provided through the data providing service connected to the decentral network as described in the context of FIG. 4. A curable composition producer may produce a curable composition from the input material(s) provided to the chemical production network. A curable composition producer may access the environmental attributes associated with the input material through a data consuming service connected to the decentral network as described in the context of FIG. 4. A curable composition producer may manage the environmental attributes via the production operating system as described in the context of FIGS. 1 to 3. A curable composition producer may assign the environmental attributes associated with the input materials or environmental attributes associated with the chemical production network such as the carbon footprint, to a curable composition as described in the context of FIGS. 1 to 3. A curable composition producer may provide the environmental attributes associated with a curable composition through the data providing service connected to the decentral network as described in the context of FIG. 4. A curable composition user or the end product producer may access the environmental attributes associated with a curable composition through the data consuming service connected to the decentral network as described in the context of FIG. 4.

The respective data owners in this example may be the input material producer, the output material producer, the output material user, the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service may comprise computer-executable instructions for accessing and/or processing data, such as a curable composition data associated with the data owner.

The data providing service may comprise computer-executable instructions for providing and/or processing data, such as a curable composition data associated with the data owner for accessing and/or processing by the data consuming service.

In the example of FIG. 4 the decentral identifier may relate to the end product. Such decentral identifier may be provided to the value chain participants. Via the end product specific decentral identifier data associated with the end product produced from a curable composition may be gathered across the production chain and assigned to the end product specific decentral identifier. For example, the one or more environmental attribute(s) associated with the end product may be derived from the environmental attribute(s) associated with a curable composition, the input material or any other product entity present in the value chain of the end product.

This way the environmental attributes of input materials, a curable composition and any products produced from a curable composition may be tracked through the value chain up to the end product. By tracking the environmental attributes of materials in such way the information can be made transparent across the value chain while the information flow can be controlled by the participants in the supply chain. In addition, the environmental attributes can be handled according to the individual participants needs by production operating systems as described in the context of FIGS. 1 to 3. Overall, such tracking enables tracking of positive environmental impact by individual supply chain participants, which makes positive environmental impacts transparent and attributable to individual supply chain participants.

FIG. 6 illustrates schematically an example of a chemical production network for producing an aqueous polymer dispersion based product associated with the composition passport, or a passport providing regulatory information or a digital asset. The example of FIG. 6 is an illustrative example and should not be considered limiting.

In the example of FIG. 6 fossil feedstock, bio feedstock and recycled feedstock may be provided to the chemical production network for producing as an example a product containing acrylic monomers as a radiation curable product (such as an ester of acrylic acid with a polyhydric alcohol like TMP, Glycerol, Pentaerytritol, ethylen- or polyethlyen glycol (called “radiation curable product”)), said acrylic monomers may be the input materials in this example as mixed feedstock. The radiation curable product may be used as component in a formulation containing at least one further component such as the other formulation compounds described before. By variation of these other formulation compounds different formulations may be produced. For example, using pigments, formulation additives and fillers as other formulation compounds, paints, printing inks or resins may be produced.

In the example of FIG. 6 the chemical description and concentration of impurities in acrylic monomers as input material may be tracked. As described in the context of FIG. 2, the chemical description and concentration of impurities in acrylic monomers as input material at the start of the “radiation curable product” production chain may be registered via the production operating system 106. Here the chemical description and concentration of impurities in acrylic monomers as input material may be allocated to the balancing account 112 for chemical description and concentration of impurities in acrylic monomers as input material. The environmental attribute of chemical description and concentration of impurities in acrylic monomers as input material is hence detached from the mass flows of the chemical processing in the chemical production network 102 as illustrated in FIG. 6.

For attribution of the chemical description and concentration of impurities in acrylic monomers as input material used to produce the radiation curable compound, the chemical description and concentration of impurities in acrylic monomers as input material may be determined. The chemical description and concentration of impurities in acrylic monomers as input material attributable to “radiation curable product” production may be based on mass conservation attributable to the produced “radiation curable product”. For example, only half of the chemical description and concentration of impurities in acrylic monomers as input material may be attributable to the “radiation curable product” and the other half may be attributable to other output products resulting from the radiation curable compound as input material. The environmental attribute chemical description and concentration of impurities in acrylic monomers as input material may be attributed to such extend to the “radiation curable product”.

In the system shown in FIG. 2, the environmental attribute associated with the production of “radiation curable product” may be attributed to the produced “radiation curable product” by associating the decentral ID to the “radiation curable product” and assigning the environmental attribute to the decentral ID. By linking the ID and the environmental attribute, the environmental attribute may be uniquely linked to the produced “radiation curable product”. The “radiation curable product” may be delivered to the “radiation curable product” user. The packaging such as lose bags with “radiation curable product” may include a QR-code. The decentral ID may be included or encoded to the QR code. This way the “radiation curable product” user may access the environmental attributes associated to the “radiation curable product” via the ID based protocol described in the context of FIGS. 4 and 5.

Similarly to this example the chemical production network for producing “radiation curable product's associated with the digital asset may be based on the method illustrated in FIG. 3 for carbon footprints.

1.-15. (canceled)

16. A system for producing a curable composition associated with a digital asset, the system comprising:
a chemical production network configured to produce a curable composition wherein a curable composition is produced from one or more input material(s) through one or more chemical process(s) of the chemical production network, wherein the one or more input material(s) and/or the one or more chemical process(s) are associated with environmental attribute(s);
a production operating apparatus configured to generate the digital asset by providing a decentral identifier associated with the produced a curable composition and one or more environmental attribute(s) of the one or more input material(s) and/or the one or more chemical process(s);
linking the decentral identifier and the environmental attribute(s);
a chemical production network or system configured to provide each produced a curable composition in association with the digital asset.

17. The system of claim 16, wherein a curable composition associated with the digital asset is comprising at least one compound selected from the group of radiation curable compounds, (poly) isocyanate compounds or epoxide groups containing compounds.

18. The system of claim 16, wherein the digital asset of a curable composition includes information of impurities related to the input material(s).

19. The system of claim 16, wherein the one or more environmental attribute(s) associated with a curable composition are provided from at least one balancing account configured to store environmental attribute(s) associated with input material(s).

20. The system of claim 16, wherein the one or more environmental attribute(s) are associated with at least one property related to the environmental impact of the one or more input material(s) and/or the chemical process(s).

21. The system of claim 16, wherein the production operating apparatus is configured to gather environmental attributes associated with the produced a curable composition before, during and/or after production of a curable composition by the chemical production network.

22. The system of claim 16, wherein the environmental attribute(s) associated with a curable composition produced through chemical processes from one or more input material(s) provided to the chemical production network include the environmental attribute(s) associated with the input material(s), the chemical process(es) and/or the chemical production network(s).

23. The system of claim 16, wherein the environmental attribute(s) associated with input material(s) are provided before, during and/or after production of a curable composition by the chemical production network, wherein the environmental attribute(s) associated with input material(s) are allocated to at least one balancing account before, during and/or after production of a curable composition by the chemical production network.

24. The system of claim 16, wherein the environmental attribute(s) associated with the produced a curable composition relate to environmental properties generated from process data associated with the chemical processing of the input material(s) and/or energy data associated with the energy consumption of the chemical processing, wherein the environmental attribute(s) associated with the produced a curable composition are generated before, during and/or after production of a curable composition by the chemical production network.

25. A method for producing a curable composition associated with a digital asset, wherein the method comprises:
producing a curable composition from one or more input material(s) through one or more chemical process(s) of a chemical production network, wherein the one or more input material(s) and/or the one or more chemical process(s) are associated with environmental attribute(s);
generating the digital asset by
providing a decentral identifier associated with the produced a curable composition and one or more environmental attribute(s) associated with the one or more input material(s) and/or the one or more chemical process(s);
linking the decentral identifier and the one or more environmental attribute(s);
providing the produced a curable composition in association with the digital asset.

26. A curable composition associated with a digital asset including a decentral identifier associated with a curable composition and linked to one or more environmental attribute(s) of the one or more input material(s) and/or the one or more chemical process(s) used to produce a curable composition.

27. A curable composition of claim 26, wherein the curable composition is comprising at least one compound selected from the group consisting of radiation curable compounds, (poly) isocyanate compounds and epoxide group containing compounds.

28. A method for generating a digital asset associated with a curable composition, wherein a curable composition is produced from one or more input material(s) through one or more chemical process(s) of a chemical production network, wherein the one or more input material(s) and/or the one or more chemical process(s) are associated with environmental attribute(s), the method comprising:
providing a decentral identifier associated with the produced a curable composition and one or more environmental attribute(s) of the one or more input material(s) and/or the one or more chemical process(s) used to produce a curable composition;
linking the decentral identifier and the environmental attribute(s);
providing the digital asset in association with the produced a curable composition, wherein the environmental attribute(s) associated with a curable composition is made accessible to a user of a curable composition through the digital asset.

29. A digital asset as generated according to the method of claim 28.

30. A method comprising using the digital asset generated according to the method of claim 28 and producing a product from a curable composition associated with the digital asset.
